SEQUENCE LISTING (23 sequences)

SEQ ID NO: 1 - protein, 239 aa, Escherichia coli
MVIKAQSPAGFAEEYIIESIWNNRFPPGTILPAERELSELIGVTRTTLREVLQRLARDGWLTIQHGKPTKVNNFWETSGLNILETLARLDHESVPQLIDNLLSVRTNISTIFIRTAFRQHPDKAQEVLATANEVADHADAFAELDYNIFRGLAFASGNPIYGLILNGMKGLYTRIGRHYFANPEARSLALGFYHKLSALCSEGAHDQVYETVRRYGHESGEIWHRMQKNLPGDLAIQGR

SEQ ID NO: 2 - protein, 473 aa, Marinobacter hydrocarbonoclasticus
MKRLGTLDASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGAKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTQYTMLLMSPYILQLMSGLGGRMRPVFNVTISNVPGPEGTLYYEGARLEAMYPVSLIAHGGALNITCLSYAGSLNFGFTGCRDTLPSMQKLAVYTGEALDELESLILPPKKRARTRK

SEQ ID NO: 3 - protein, 455 aa, Marinobacter hydrocarbonoclasticus
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSAMRMATRMLSENPDEHGMPPIWDLPCLSRDRGESDGHSLWRSVTHLLGLSDRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQDAGERLLKIHHGMEEAKQRYRHMSPEEIVNYTALTLAPAAFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 4 - protein, 457 aa, Alcanivorax borkumensis
MKALSPVDQLFLWLEKRQQPMHVGGLQLFSFPEGAGPKYVSELAQQMRDYCHPVAPFNQRLTRRLGQYYWTRDKQFDIDHHFRHEALPKPGRIRELLSLVSAEHSNLLDRERPMWEAHLIEGIRGRQFALYYKIHHSVMDGISAMRIASKTLSTDPSEREMAPAWAFNTKKRSRSLPSNPVDMASSMARLTASISKQAATVPGLAREVYKVTQKAKKDENYVSIFQAPDTILNNTITGSRRFAAQSFPLPRLKVIAKAYNCTINTVVLSMCGHALREYLISQHALPDEPLIAMVPMSLRQDDSTGGNQIGMILANLGTHICDPANRLRVIHDSVEEAKSRFSQMSPEEILNFTALTMAPTGLNLLTGLAPKWRAFNVVISNIPGPKEPLYWNGAQLQGVYPVSIALDRIALNITLTSYVDQMEFGLIACRRTLPSMQRLLDYLEQSIRELEIGAGIK

SEQ ID NO: 5 - protein, 451 aa, Alcanivorax borkumensis
MARKLSIMDSGWLMMETRETPMHVGGLALFAIPEGAPEDYVESIYRYLVDVDSICRPFNQKIQSHLPLYLDATWVEDKNFDIDYHVRHSALPRPGRVRELLALVSRLHAQRLDPSRPLWESYLIEGLEGNRFALYTKMHHSMVDGVAGMHLMQSRLATCAEDRLPAPWSGEWDAEKKPRKSRGAAAANAGMKGTMNNLRRGGGQLVDLLRQPKDGNVKTIYRAPKTQLNRRVTGARRFAAQSWSLSRIKAAGKQHGGTVNDIFLAMCGGALRRYLLSQDALSDQPLVAQVPVALRSADQAGEGGNAITTVQVSLGTHIAQPLNRLAAIQDSMKAVKSRLGDMQKSEIDVYTVLTNMPLSLGQVTGLSGRVSPMFNLVISNVPGPKETLHLNGAEMLATYPVSLVLHGYALNITVVSYKNSLEFGVIGCRDTLPHIQRFLVYLEESLVELEP

SEQ ID NO: 6 - protein, 455 aa, Marinobacter aquaeolei
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSAMRMATRMLSENPDEHGMPPIWDLPCLSRDRGESDGHSLWRSVTHLLGLSGRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQEAGERLLKIHHGMEEAKQRYRHMSPEEIVNYTALTLAPAAFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 7 - protein, 473 aa, Marinobacter aquaeolei
MKRLGTLDASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGAKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTQYTMLLMSPYILQLMSGLGGRMRPVFNVTISNVPGPEDTLYYEGARLEAMYPVSLIAHGGALNITCLSYAGSLNFGFTGCRDTLPSMQKLAVYTGEALDELESLILPPKKRARTRK

SEQ ID NO: 8 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 9 - protein, 341 aa, Acinetobacter baylyi
MATTNVIHAYAAMQAGEALVPYSFDAGELQPHQVEVKVEYCGLCHSDVSVLNNEWHSSVYPVVAGHEVIGTITQLGSEAKGLKIGQRVGIGWTAESCQACDQCISGQQVLCTGENTATIIGHAGGFADKVRAGWQWVIPLPDELDPTSAGPLLCGGITVFDPILKHQIQAIHHVAVIGIGGLGHMAIKLLKAWGCEITAFSSNPNKTDELKAMGADHVVNSRDDAEIKSQQGKFDLLLSTVNVPLNWNAYLNTLAPNGTFHFLGVVMEPIPVPVGALLGGAKSLTASPTGSPAALRKLLEFAARKNIAPQIEMYPMSELNEAIERLHSGQARYRIVLKADF

SEQ ID NO: 10 - protein, 339 aa, Escherichia coli
MSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADF

SEQ ID NO: 11 - protein, 208 aa, Escherichia coli
MMNFNNVFRWHLPFLFLVLLTFRAAAADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 12 - protein, 209 aa, Escherichia coli
MVDMKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASEIQTDAGFLDYQIISWNKQQVIIHRENEMFAVHWQIKEKIVITLCQHD

SEQ ID NO: 13 - DNA, 12397 bp, synthetic construct
  misc_feature 1..12397: note = source = /note="Description of Artificial Sequence: Synthetic polynucleotide"
  source 1..12397: mol_type = other DNA
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttagaattccataattgctgttaggagatatatatggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggctgtgacggtttgcgtggttttcagccacagcaaaccgagcaaacgctgcgccagattttgcaggatgtcaaagccgccaacgctcttccattgttaatgcaaatacgtctgccttacaactatggtcgtcgttataatgaagcctttagcgccatttaccccaaactcgccaaagagtttgatgttccgctgctgcccttttttatggaagaggtctgcctcaagccacaatggatgcaggatgacggtattcatcccaaccgcgacgcccagccgtttattgccgactggatggcgaagcagttgcagcctttaaccaatcatgactcataagcttctaaggaaataataggagattgaaaatggcaacaactaatgtgattcatgcttatgctgcaatgcaggcaggtgaagcactcgtgccttattcgtttgatgcaggcgaactgcaaccacatcaggttgaagttaaagtcgaatattgtgggctgtgccattccgatgtctcggtactcaacaacgaatggcattcttcggtttatccagtcgtggcaggtcatgaagtgattggtacgattacccaactgggaagtgaagccaaaggactaaaaattggtcaacgtgttggtattggctggacggcagaaagctgtcaggcctgtgaccaatgcatcagtggtcagcaggtattgtgcacgggcgaaaataccgcaactattattggtcatgctggtggctttgcagataaggttcgtgcaggctggcaatgggtcattcccctgcccgacgaactcgatccgaccagtgctggtcctttgctgtgtggcggaatcacagtatttgatccaattttaaaacatcagattcaggctattcatcatgttgctgtgattggtatcggtggtttgggacatatggccatcaagctacttaaagcatggggctgtgaaattactgcgtttagttcaaatccaaacaaaaccgatgagctcaaagctatgggggccgatcacgtggtcaatagccgtgatgatgccgaaattaaatcgcaacagggtaaatttgatttactgctgagtacagttaatgtgcctttaaactggaatgcgtatctaaacacactggcacccaatggcactttccattttttgggcgtggtgatggaaccaatccctgtacctgtcggtgcgctgctaggaggtgccaaatcgctaacagcatcaccaactggctcgcctgctgccttacgtaagctgctcgaatttgcggcacgtaagaatatcgcacctcaaatcgagatgtatcctatgtcggagctgaatgaggccatcgaacgcttacattcgggtcaagcacgttatcggattgtacttaaagccgatttttaacctagggataatagaggttaagagcggccagatgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaaactttatcgaaatggccatccattcttggtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

SEQ ID NO: 14 - DNA, 3227 bp, synthetic construct
  misc_feature 1..3227: note = source = /note="Description of Artificial Sequence: Synthetic polynucleotide"
  source 1..3227: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttgtgcatccgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttgaacaataaaattaatataaatcagcaacttaaatagcctctaaggttttaagttttataagaaaaaaaagaatatataaggcttttaaagctagcttttaaggtttcaccatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaagacagctgtctcttatacacatctcaaccctgaagctcttgttggctagtgcgtagtcgttggcaagctttccgctgtttctgcattcttacgttttaggatgcatatggcggccgcataacttcgtatagcatacattatacgaagttatctagagttgcatgcctgcaggtccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgactctagataacttcgtatagcatacattatacgaagttatggatccagcttatcgataccgtcaaacaaatcataaaaaatttatttgctttcaggaaaatttttctgtataatagattcaattgcgatgacgacgaacacgcattaaggaggtgaagagctcgaattcgagccaatatgcgagaacacccgagaaaattcatcgatgatggttgagatgtgtataagagacagctgtcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 15 - DNA, 40 bp, synthetic construct
  misc_feature 1..40: note = source = /note="Description of Artificial Sequence: Synthetic primer"
  source 1..40: mol_type = other DNA
gcagttattggtgcccttaaacgcctggttgctacgcctg

SEQ ID NO: 16 - DNA, 34 bp, synthetic construct
  misc_feature 1..34: note = source = /note="Description of Artificial Sequence: Synthetic primer"
  source 1..34: mol_type = other DNA
cccagggcttcccggtatcaacagggacaccagg

SEQ ID NO: 17 - DNA, 25 bp, synthetic construct
  misc_feature 1..25: note = source = /note="Description of Artificial Sequence: Synthetic primer"
  source 1..25: mol_type = other DNA
atggtcattaaggcgcaaagcccgg

SEQ ID NO: 18 - DNA, 53 bp, synthetic construct
  misc_feature 1..53: note = source = /note="Description of Artificial Sequence: Synthetic primer"
  source 1..53: mol_type = other DNA
gagaccccacactaccatcctcgagttatcgcccctgaatggctaaatcaccc

SEQ ID NO: 19 - DNA, 27 bp, synthetic construct
  misc_feature 1..27: note = source = /note="Description of Artificial Sequence: Synthetic primer"
  source 1..27: mol_type = other DNA
ctcgaggatggtagtgtggggtctccc

SEQ ID NO: 20 - DNA, 69 bp, synthetic construct
  misc_feature 1..69: note = source = /note="Description of Artificial Sequence: Synthetic primer"
  source 1..69: mol_type = other DNA
gagaccgtttctcgaatttaaatatgatacgctcgagcttcgtctgtttctactggtattggcacaaac

SEQ ID NO: 21 - DNA, 65 bp, synthetic construct
  misc_difference 16: note = a, c, t, g, unknown or other
  misc_difference 21: note = a, c, t, g, unknown or other
  misc_difference 27: note = a, c, t, g, unknown or other
  misc_difference 34..40: note = a, c, t, g, unknown or other
  misc_feature 1..65: note = source = /note="Description of Artificial Sequence: Synthetic primer"
  source 1..65: mol_type = other DNA
tgaaagattaaatttnhharnddhddnwaggagnnnnnnnatggtcattaaggcgcaaagcccgg

SEQ ID NO: 22 - DNA, 12206 bp, synthetic construct
  misc_feature 1..12206: note = source = /note="Description of Artificial Sequence: Synthetic polynucleotide"
  source 1..12206: mol_type = other DNA
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttagaattctttagcgttaagaaggagatatatatggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgccttacaactatggtcgtcgttataatgaagcctttagcgccatttaccccaaactcgccaaagagtttgatgttccgctgctgcccttttttatggaagaggtctgcctcaagccacaatggatgcaggatgacggtattcatcccaaccgcgacgcccagccgtttattgccgactggatggcgaagcagttgcagcctttagtaaatcatgactcataagcttctaaggaaataataggagattgaaaatggcaacaactaatgtgattcatgcttatgctgcaatgcaggcaggtgaagcactcgtgccttattcgtttgatgcaggcgaactgcaaccacatcaggttgaagttaaagtcgaatattgtgggctgtgccattccgatgtctcggtactcaacaacgaatggcattcttcggtttatccagtcgtggcaggtcatgaagtgattggtacgattacccaactgggaagtgaagccaaaggactaaaaattggtcaacgtgttggtattggctggacggcagaaagctgtcaggcctgtgaccaatgcatcagtggtcagcaggtattgtgcacgggcgaaaataccgcaactattattggtcatgctggtggctttgcagataaggttcgtgcaggctggcaatgggtcattcccctgcccgacgaactcgatccgaccagtgctggtcctttgctgtgtggcggaatcacagtatttgatccaattttaaaacatcagattcaggctattcatcatgttgctgtgattggtatcggtggtttgggacatatggccatcaagctacttaaagcatggggctgtgaaattactgcgtttagttcaaatccaaacaaaaccgatgagctcaaagctatgggggccgatcacgtggtcaatagccgtgatgatgccgaaattaaatcgcaacagggtaaatttgatttactgctgagtacagttaatgtgcctttaaactggaatgcgtatctaaacacactggcacccaatggcactttccattttttgggcgtggtgatggaaccaatccctgtacctgtcggtgcgctgctaggaggtgccaaatcgctaacagcatcaccaactggctcgcctgctgccttacgtaagctgctcgaatttgcggcacgtaagaatatcgcacctcaaatcgagatgtatcctatgtcggagctgaatgaggccatcgaacgcttacattcgggtcaagcacgttatcggattgtacttaaagccgatttttaacctaggatcagtatctggtaggagatcacggatgaaacgtgcagtgattactggcctgggcattgtttccagcatcggtaataaccagcaggaagtcctggcatctctgcgtgaaggacgttcagggatcactttctctcaggagctgaaggattccggcatgcgtagccacgtctggggcaacgtaaaactggataccactggcctcattgaccgcaaagttgtgcgctttatgagcgacgcatccatttatgcattcctttctatggagcaggcaatcgctgatgcgggcctctctccggaagcttaccagaataacccgcgcgttggcctgattgcaggttccggcggcggctccccgcgtttccaggtgttcggcgctgacgcaatgcgcggcccgcgcggcctgaaagcggttggcccgtatgtggtcaccaaagcgatggcatccggcgtttctgcctgcctcgccaccccgtttaaaattcatggcgttaactactccatcagctccgcgtgtgcgacttccgcacactgtatcggtaacgcagtagagcagatccaactgggcaaacaggacatcgtgtttgctggcggcggcgaagagctgtgctgggaaatggcttgcgaattcgacgcaatgggtgcgctgtctactaaatacaacgacaccccggaaaaagcctcccgtacttacgacgctcaccgtgacggtttcgttatcgctggcggcggcggtatggtagtggttgaagagctggaacacgcgctggcgcgtggtgctcacatctatgctgaaatcgttggctacggcgcaacctctgatggtgcagacatggttgctccgtctggcgaaggcgcagtacgctgcatgaagatggcgatgcatggcgttgataccccaatcgattacctgaactcccacggtacttcgactccggttggcgacgtgaaagagctggcagctatccgtgaagtgttcggcgataagagcccggcgatttctgcaaccaaagggatgaccggtcactctctgggcgctgctggcgtacaggaagctatctactctctgctgatgctggaacacggctttatcgccccgagcatcaacattgaagagctggacgagcaggctgcgggtctgaacatcgtgaccgaaacgaccgatcgcgaactgaccaccgttatgtctaacagcttcggcttcggcggcaccaacgccacgctggtaatgcgcaagctgaaagattaaatttaaatgctatgtctcgagaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

SEQ ID NO: 23 - DNA, 14893 bp, synthetic construct
  misc_feature 1..14893: note = source = /note="Description of Artificial Sequence: Synthetic polynucleotide"
  source 1..14893: mol_type = other DNA
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttagaattctttagcgttaagaaggagatatatatggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgccttacaactatggtcgtcgttataatgaagcctttagcgccatttaccccaaactcgccaaagagtttgatgttccgctgctgcccttttttatggaagaggtctgcctcaagccacaatggatgcaggatgacggtattcatcccaaccgcgacgcccagccgtttattgccgactggatggcgaagcagttgcagcctttagtaaatcatgactcataagcttctaaggaaataataggagattgaaaatggcaacaactaatgtgattcatgcttatgctgcaatgcaggcaggtgaagcactcgtgccttattcgtttgatgcaggcgaactgcaaccacatcaggttgaagttaaagtcgaatattgtgggctgtgccattccgatgtctcggtactcaacaacgaatggcattcttcggtttatccagtcgtggcaggtcatgaagtgattggtacgattacccaactgggaagtgaagccaaaggactaaaaattggtcaacgtgttggtattggctggacggcagaaagctgtcaggcctgtgaccaatgcatcagtggtcagcaggtattgtgcacgggcgaaaataccgcaactattattggtcatgctggtggctttgcagataaggttcgtgcaggctggcaatgggtcattcccctgcccgacgaactcgatccgaccagtgctggtcctttgctgtgtggcggaatcacagtatttgatccaattttaaaacatcagattcaggctattcatcatgttgctgtgattggtatcggtggtttgggacatatggccatcaagctacttaaagcatggggctgtgaaattactgcgtttagttcaaatccaaacaaaaccgatgagctcaaagctatgggggccgatcacgtggtcaatagccgtgatgatgccgaaattaaatcgcaacagggtaaatttgatttactgctgagtacagttaatgtgcctttaaactggaatgcgtatctaaacacactggcacccaatggcactttccattttttgggcgtggtgatggaaccaatccctgtacctgtcggtgcgctgctaggaggtgccaaatcgctaacagcatcaccaactggctcgcctgctgccttacgtaagctgctcgaatttgcggcacgtaagaatatcgcacctcaaatcgagatgtatcctatgtcggagctgaatgaggccatcgaacgcttacattcgggtcaagcacgttatcggattgtacttaaagccgatttttaacctaggatcagtatctggtaggagatcacggatgaaacgtgcagtgattactggcctgggcattgtttccagcatcggtaataaccagcaggaagtcctggcatctctgcgtgaaggacgttcagggatcactttctctcaggagctgaaggattccggcatgcgtagccacgtctggggcaacgtaaaactggataccactggcctcattgaccgcaaagttgtgcgctttatgagcgacgcatccatttatgcattcctttctatggagcaggcaatcgctgatgcgggcctctctccggaagcttaccagaataacccgcgcgttggcctgattgcaggttccggcggcggctccccgcgtttccaggtgttcggcgctgacgcaatgcgcggcccgcgcggcctgaaagcggttggcccgtatgtggtcaccaaagcgatggcatccggcgtttctgcctgcctcgccaccccgtttaaaattcatggcgttaactactccatcagctccgcgtgtgcgacttccgcacactgtatcggtaacgcagtagagcagatccaactgggcaaacaggacatcgtgtttgctggcggcggcgaagagctgtgctgggaaatggcttgcgaattcgacgcaatgggtgcgctgtctactaaatacaacgacaccccggaaaaagcctcccgtacttacgacgctcaccgtgacggtttcgttatcgctggcggcggcggtatggtagtggttgaagagctggaacacgcgctggcgcgtggtgctcacatctatgctgaaatcgttggctacggcgcaacctctgatggtgcagacatggttgctccgtctggcgaaggcgcagtacgctgcatgaagatggcgatgcatggcgttgataccccaatcgattacctgaactcccacggtacttcgactccggttggcgacgtgaaagagctggcagctatccgtgaagtgttcggcgataagagcccggcgatttctgcaaccaaagggatgaccggtcactctctgggcgctgctggcgtacaggaagctatctactctctgctgatgctggaacacggctttatcgccccgagcatcaacattgaagagctggacgagcaggctgcgggtctgaacatcgtgaccgaaacgaccgatcgcgaactgaccaccgttatgtctaacagcttcggcttcggcggcaccaacgccacgctggtaatgcgcaagctgaaagattaaatttctaaaattcagttaggaggtatttgatggtcattaaggcgcaaagcccggcgggtttcgcggaagagtacattattgaaagtatctggaataaccgcttccctcccgggactattttgcccgcagaacgtgaactttcagaattaattggcgtaacgcgtactacgttacgtgaagtgttacagcgtctggcacgagatggctggttgaccattcaacatggcaagccgacgaaggtgaataatttctgggaaacttccggtttaaatatccttgaaacactggcgcgactggatcacgaaagtgtgccgcagcttattgataatttgctgtcggtgcgtaccaatatttccactatttttattcgcaccgcgtttcgtcagcatcccgataaagcgcaggaagtgctggctaccgctaatgaagtggccgatcacgccgatgcctttgccgagctggattacaacatattccgcggcctggcgtttgcttccggcaacccgatttacggtctgattcttaacgggatgaaagggctgtatacgcgtattggtcgtcactatttcgccaatccggaagcgcgcagtctggcgctgggcttctaccacaaactgtcggcgttgtgcagtgaaggcgcgcacgatcaggtgtacgaaacagtgcgtcgctatgggcatgagagtggcgagatttggcaccggatgcagaaaaatctgccgggtgatttagccattcaggggcgataactcgaggatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcaagatccccttattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgagcttcaaaagcgctctgaagttcctatactttctagagaataggaacttcgaactgcaggtcgacggatccccggaattaattctcatgtttgacagcttatcactgatcagtgaattaatggcgatgacgcatcctcacgataatatccgggtaggcgcaatcactttcgtctctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagctcgagcgtatcatatttaaattcgagaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

(19) United States
(12) Patent Application Publication
(10) Pub. No.: US 2025/0092355 A1
(43) Pub. Date: Mar. 20, 2025

(54) Methods And Compositions For Improved Production Of Fatty Acids And Derivatives Thereof

(71) Applicant: Genomatica, Inc., San Diego, CA (US)

(72) Inventors: Derek L. GREENFIELD, South San Francisco, CA (US); Louis G. HOM, San Carlos, CA (US); Fernando A. SANCHEZ-RIERA, South San Francisco, CA (US); Zhihao HU, Castro Valley, CA (US); Vikranth ARLAGADDA, San Bruno, CA (US); Eli S. GROBAN, South San Francisco, CA (US); Scott A. FRYKMAN, San Francisco, CA (US)

(21) Appl. No.: 18/816,019

(22) Filed: Aug. 27, 2024

Related U.S. Application Data
(63) Continuation of application No. 17/350,690, filed on Jun. 17, 2021, now abandoned.
Application No. 17/350,690 is a continuation of application No. 14/007,857, filed on Jan. 22, 2014, now Pat. No. 11,066,682.
(60) Provisional application No. 61/470,989, filed on Apr. 1, 2011.

(51) Int. Cl. C12N 1/20; C07C 31/125; C07C 33/02; C07C 53/126; C07K 14/195; C07K 14/24; C07K 14/245; C07K 14/255; C07K 14/26; C12P 7/6409

(52) U.S. Cl. CPC C12N 1/20; C07C 31/125; C07C 33/02; C07C 53/126; C07K 14/195; C07K 14/24; C07K 14/245; C07K 14/255; C07K 14/26; C12P 7/6409; C12Y 207/08007; Y02E 50/10

(57) ABSTRACT

The invention relates to compositions and methods, including polynucleotide sequences, amino acid sequences, and engineered host cells for producing fatty acids and derivates of fatty acids such as acyl-CoA, terminal olefins, fatty aldehydes, fatty alcohols, alkanes, alkenes, wax esters, ketones and internal olefins through altered expression of the transcription factor, fadR.

### CROSS-REFERENCE TO RELATED APPLICATIONS

This application is a continuation of U.S. application Ser. No. 17/350,690 filed Jun. 17, 2021 which is a continuation of U.S. application Ser. No. 14/007,857 filed Jan. 22, 2014 which is a national phase application of PCT Application No.: PCT/US2012/031881 filed 2 Apr. 2012 which claims the benefit of U.S. Application No. 61/470,989, filed Apr. 1, 2011. Each application recited above is incorporated by reference herein in its entirety.

### REFERENCE TO AN ELECTRONIC SEQUENCE LISTING

This application contains references to amino acid sequences and/or nucleic acid sequences which have been submitted concurrently herewith as the sequence listing.xml file entitled “ST26_SL_Conversion_21_Aug_2024.xml”, file size 74,000 Bytes (B), created on 21 Aug. 2024. The aforementioned sequence listing is hereby incorporated by reference in its entirety.

### BACKGROUND OF THE INVENTION

Crude petroleum is a limited, natural resource found in the Earth in liquid, gaseous, and solid forms. Although crude petroleum is a valuable resource, it is discovered and extracted from the Earth at considerable financial and environmental costs. Moreover, in its natural form, crude petroleum extracted from the Earth has few commercial uses. Crude petroleum is a mixture of hydrocarbons (e.g., paraffins (or alkanes), olefins (or alkenes), alkynes, napthenes (or cycloalkanes), aliphatic compounds, aromatic compounds, etc.) of varying length and complexity. In addition, crude petroleum contains other organic compounds (e.g., organic compounds containing nitrogen, oxygen, sulfur, etc.) and impurities (e.g., sulfur, salt, acid, metals, etc.). Hence, crude petroleum must be refined and purified at considerable cost before it can be used commercially.

Crude petroleum is also a primary source of raw materials for producing petrochemicals. The two main classes of raw materials derived from petroleum are short chain olefins (e.g., ethylene and propylene) and aromatics (e.g., benzene and xylene isomers). These raw materials are derived from longer chain hydrocarbons in crude petroleum by cracking it at considerable expense using a variety of methods, such as catalytic cracking, steam cracking, or catalytic reforming. These raw materials can be used to make petrochemicals such as monomers, solvents, detergents, and adhesives, which otherwise cannot be directly refined from crude petroleum.

Petrochemicals, in turn, can be used to make specialty chemicals, such as plastics, resins, fibers, elastomers, pharmaceuticals, lubricants, and gels. Particular specialty chemicals that can be produced from petrochemical raw materials include fatty acids, hydrocarbons (e.g., long chain, branched chain, saturated, unsaturated, etc.), fatty aldehydes, fatty alcohols, esters, ketones, lubricants, etc.

Due to the inherent challenges posed by petroleum, there is a need for a renewable petroleum source that does not need to be explored, extracted, transported over long distances, or substantially refined like crude petroleum. There is also a need for a renewable petroleum source which can be produced economically without creating the type of environmental damage produced by the petroleum industry and the burning of petroleum-based fuels. For similar reasons, there is also a need for a renewable source of chemicals which are typically derived from petroleum.

One method of producing renewable petroleum is by engineering microorganisms to produce renewable petroleum products. Some microorganisms have long been known to possess a natural ability to produce petroleum products (e.g., yeast to produce ethanol). More recently, the development of advanced biotechnologies has made it possible to metabolically engineer an organism to produce bioproducts and biofuels. Bioproducts (e.g., chemicals) and biofuels (e.g., biodiesel) are renewable alternatives to petroleum-based chemicals and fuels, respectively. Bioproducts and biofuels can be derived from renewable sources, such as plant matter, animal matter, and organic waste matter, which are collectively known as biomass.

Biofuels can be substituted for any petroleum-based fuel (e.g., gasoline, diesel, aviation fuel, heating oil, etc.), and offer several advantages over petroleum-based fuels. Biofuels do not require expensive and risky exploration or extraction. Biofuels can be produced locally and therefore do not require transportation over long distances. In addition, biofuels can be made directly and require little or no additional refining. Furthermore, the combustion of biofuels causes less of a burden on the environment since the amount of harmful emissions (e.g., green house gases, air pollution, etc.) released during combustion is reduced as compared to the combustion of petroleum-based fuels. Moreover, biofuels maintain a balanced carbon cycle because biofuels are produced from biomass, a renewable, natural resource. Although combustion of biofuels releases carbon (e.g., as carbon dioxide), this carbon will be recycled during the production of biomass (e.g., the cultivation of crops), thereby balancing the carbon cycle, which is not achieved with the use of petroleum based fuels.

Biologically derived chemicals offer similar advantages over petrochemicals that biofuels offer over petroleum-based fuels. In particular, biologically derived chemicals can be converted from biomass to the desired chemical product directly without extensive refining, unlike petrochemicals, which must be produced by refining crude petroleum to recover raw materials which are then processed further into the desired petrochemical.

Hydrocarbons have many commercial uses. For example, shorter chain alkanes are used as fuels. Methane and ethane are the main constituents of natural gas. Longer chain alkanes (e.g., from five to sixteen carbons) are used as transportation fuels (e.g., gasoline, diesel, or aviation fuel). Alkanes having more than sixteen carbon atoms are important components of fuel oils and lubricating oils. Even longer alkanes, which are solid at room temperature, can be used, for example, as a paraffin wax. Alkanes that contain approximately thirty-five carbons are found in bitumen, which is used for road surfacing. In addition, longer chain alkanes can be cracked to produce commercially useful shorter chain hydrocarbons.

Like short chain alkanes, short chain alkenes are used in transportation fuels. Longer chain alkenes are used in plastics, lubricants, and synthetic lubricants. In addition, alkenes are used as a feedstock to produce alcohols, esters, plasticizers, surfactants, tertiary amines, enhanced oil recovery agents, fatty acids, thiols, alkenylsuccinic anhydrides, epoxides, chlorinated alkanes, chlorinated alkenes, waxes, fuel additives, and drag flow reducers.

Esters have many commercial uses. For example, biodiesel, an alternative fuel, is comprised of esters (e.g., fatty acid methyl ester, fatty acid ethyl esters, etc.). Some low molecular weight esters are volatile with a pleasant odor which makes them useful as fragrances or flavoring agents. In addition, esters are used as solvents for lacquers, paints, and varnishes. Furthermore, some naturally occurring substances, such as waxes, fats, and oils are comprised of esters. Esters are also used as softening agents in resins and plastics, plasticizers, flame retardants, and additives in gasoline and oil. In addition, esters can be used in the manufacture of polymers, films, textiles, dyes, and pharmaceuticals.

Aldehydes are used to produce many specialty chemicals. For example, aldehydes are used to produce polymers, resins (e.g., Bakelite), dyes, flavorings, plasticizers, perfumes, pharmaceuticals, and other chemicals, some of which may be used as solvents, preservatives, or disinfectants. In addition, certain natural and synthetic compounds, such as vitamins and hormones, are aldehydes, and many sugars contain aldehyde groups. Fatty aldehydes can be converted to fatty alcohols by chemical or enzymatic reduction.

Fatty alcohols have many commercial uses. Worldwide annual sales of fatty alcohols and their derivatives are in excess of U.S. $1 billion. The shorter chain fatty alcohols are used in the cosmetic and food industries as emulsifiers, emollients, and thickeners. Due to their amphiphilic nature, fatty alcohols behave as nonionic surfactants, which are useful in personal care and household products, such as, for example, detergents. In addition, fatty alcohols are used in waxes, gums, resins, pharmaceutical salves and lotions, lubricating oil additives, textile antistatic and finishing agents, plasticizers, cosmetics, industrial solvents, and solvents for fats.

Acyl-CoA synthase (ACS) esterifies free fatty acids to acyl-CoA by a two-step mechanism. The free fatty acid first is converted to an acyl-AMP intermediate (an adenylate) through the pyrophosphorolysis of ATP. The activated carbonyl carbon of the adenylate is then coupled to the thiol group of CoA, releasing AMP and the acyl-CoA final product (Shockey et al., Plant. Physiol., 129:1710-1722 (2002)).

FadR is a key regulatory factor involved in fatty acid degradation and fatty acid biosynthesis pathways (Cronan et al., Mol. Microbiol., 29(4): 937-943 (1998)). The E. coli ACS enzyme FadD and the fatty acid transport protein FadL are essential components of a fatty acid uptake system. FadL mediates transport of fatty acids into the bacterial cell, and FadD mediates formation of acyl-CoA esters. When no other carbon source is available, exogenous fatty acids are taken up by bacteria and converted to acyl-CoA esters, which can bind to the transcription factor FadR and derepress the expression of the fad genes that encode proteins responsible for fatty acid transport (FadL), activation (FadD), and β-oxidation (FadA, FadB, FadE, and FadH). When alternative sources of carbon are available, bacteria synthesize fatty acids as acyl-ACPs, which are used for phospholipid synthesis, but are not substrates for β-oxidation. Thus, acyl-CoA and acyl-ACP are both independent sources of fatty acids that can result in different end-products (Caviglia et al., J. Biol. Chem., 279(12): 1163-1169 (2004)).

There remains a need for methods and compositions for enhancing the production of biologically derived chemicals, such as fatty acids and fatty acid derivatives. This invention provides such methods and compositions. The invention further provides products derived from the fatty acids and derivatives thereof produced by the methods described herein, such as fuels, surfactants, and detergents.

### BRIEF SUMMARY OF THE INVENTION

The invention provides improved methods of producing a fatty acid or a fatty acid derivative in a host cell. The method comprises (a) providing a host cell which is genetically engineered to have an altered level of expression of a FadR polypeptide as compared to the level of expression of the FadR polypeptide in a corresponding wild-type host cell, (b) culturing the engineered host cell in a culture medium under conditions permissive for the production of a fatty acid or a fatty acid derivative, and (c) isolating the fatty acid or fatty acid derivative from the engineered host cell. As a result of this method, one or more of the titer, yield, or productivity of the fatty acid or fatty acid derivative produced by the engineered host cell is increased relative to that of the corresponding wild-type host cell.

Also provided are fatty acids and fatty acid derivatives, such as an acyl-CoA, a fatty aldehyde, a short chain alcohol, a long chain alcohol, a fatty alcohol, a hydrocarbon, or an ester, produced by the methods of the invention. Further provided are biofuel compositions and surfactant compositions comprising a fatty acid or a fatty acid derivative produced by the methods of the invention.

### BRIEF DESCRIPTION OF THE SEVERAL VIEWS OF THE DRAWING(S)

FIG. 1A-FIG. 1T is a chart of exemplary genes suitable for use in practicing the invention. Polypeptide and/or polynucleotide accession numbers are from the National Center for Biotechnology Information (NCBI) database, and enzyme EC numbers are from the Nomenclature Committee of the International Union of Biochemistry and Molecular Biology (NC-IUBMB).

FIG. 2 is a graph of fatty species production in a control E. coli strain (ALC310) or the transposon insertion strain, D288.

FIG. 3 is a diagram depicting the location of the transposon insertion in the D288 strain.

FIG. 4 is a bar graph of total fatty species (FA) titers in expression library E. coli strains having altered expression of wild-type FadR or mutant FadR [S219N] as compared to FA titers in the control E. coli strain (ALC487).

FIG. 5 is a bar graph of total fatty species (FA) titers in three separate shake flask (SF) fermentations of E. coli strain D512 having altered expression of wild-type FadR as compared to FA titers in the control ALC487 strain.

FIG. 6 is a bar graph of total fatty species yield on carbon in shake flask fermentations of the control ALC487 strain or E. coli strain D512 having altered expression of wild-type FadR.

FIG. 7 is a graph of fatty acid and fatty alcohol production and total fatty species yield in 5 L bioreactor fermentations of the control ALC487 strain fed at a glucose rate of 10 g/L/hr or the D512 strain having altered expression of wild-type FadR fed at a glucose rate of 10 g/L/hr or 15 g/L/hr. The bars represent fatty alcohol or fatty acid titer, and the circles represent total fatty species yield on carbon.

FIG. 8 is a graph of fatty acid and fatty alcohol production and total fatty species yield in shake flask fermentations of the D512 strain or a D512 strain in which the entD gene was deleted. The bars represent fatty acid or fatty alcohol titer, and the circles represent fatty acid yield.

FIG. 9 is a graph of total fatty species (fatty acids and fatty acid methyl ester (FAME)) titers and yields in two ribosome binding site (RBS) library E. coli strains having altered expression of mutant FadR [S219N] (i.e., P1A4 and P1G7) as compared to the total fatty species titers and yields in the parental E. coli strain (DAM1-pDS57) in shake flask (SF) fermentations at 32° C. The bars represent total fatty species titers after 56 hours of culture, and the squares represent total fatty species yield after 56 hours of culture.

FIG. 10 is a line graph of combined FAME and free fatty acid (FFA) titers in the parental DAM1 pDS57 strain, or RBS library strains P1A4 or P1G7 in bioreactor fermentations at several timepoints following induction of FAME and FFA production, wherein DAM1 P1A4 and DAM1 P1G7 express FadR and DAM1 pDS57 does not express FadR.

FIG. 11 is a line graph of combined FAME and FFA yields in the parental DAM1 pDS57 strain, or RBS library strains P1A4 or P1G7 in bioreactor fermentations at several time points following induction of FAME and FFA production, wherein DAM1 P1A4 and DAM1 P1G7 express FadR and DAM1 pDS57 does not express FadR.

### DETAILED DESCRIPTION OF THE INVENTION

The invention is based, at least in part, on the discovery that altering the level of expression of FadR in a host cell facilitates enhanced production of fatty acids and fatty acid derivatives by the host cell.

The invention provides improved methods of producing a fatty acid or a fatty acid derivative in a host cell. The method comprises (a) providing a host cell which is genetically engineered to have an altered level of expression of a FadR polypeptide as compared to the level of expression of the FadR polypeptide in a corresponding wild-type host cell, (b) culturing the engineered host cell in a culture medium under conditions permissive for the production of a fatty acid or a fatty acid derivative, and (c) isolating the fatty acid or fatty acid derivative from the engineered host cell. As a result of this method, one or more of the titer, yield, or productivity of the fatty acid or fatty acid derivative produced by the engineered host cell is increased relative to that of the corresponding wild-type host cell.

### Definitions

As used in this specification and the appended claims, the singular forms “a,” “an” and “the” include plural referents unless the context clearly dictates otherwise. Thus, for example, reference to “a recombinant host cell” includes two or more such recombinant host cells, reference to “a fatty alcohol” includes one or more fatty alcohols, or mixtures of fatty alcohols, reference to “a nucleic acid coding sequence” includes one or more nucleic acid coding sequences, reference to “an enzyme” includes one or more enzymes, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

Accession Numbers: Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers”), and from the UniProt Knowledgebase (UniProtKB) and Swiss-Prot databases provided by the Swiss Institute of Bioinformatics (which are identified herein as “UniProtKB Accession Numbers”).

Enzyme Classification (EC) Numbers: EC numbers are established by the Nomenclature Committee of the International Union of Biochemistry and Molecular Biology (IUBMB), description of which is available on the IUBMB Enzyme Nomenclature website on the World Wide Web. EC numbers classify enzymes according to the reaction catalyzed.

The term “FadR polypeptide” refers to a polypeptide having biological activity corresponding to that of FadR derived from E. coli MG1655 (SEQ ID NO: 1).

As used herein, the term “fatty acid or derivative thereof” means a “fatty acid” or a “fatty acid derivative.” The term “fatty acid” means a carboxylic acid having the formula RCOOH. R represents an aliphatic group, preferably an alkyl group. R can comprise between about 4 and about 22 carbon atoms. Fatty acids can be saturated, monounsaturated, or polyunsaturated. In a preferred embodiment, the fatty acid is made from a fatty acid biosynthetic pathway. A “fatty acid derivative” is a product made in part from the fatty acid biosynthetic pathway of the production host organism. “Fatty acid derivatives” includes products made in part from acyl-ACP or acyl-ACP derivatives. Exemplary fatty acid derivatives include, for example, acyl-CoA, fatty acids, fatty aldehydes, short and long chain alcohols, hydrocarbons, fatty alcohols, esters (e.g., waxes, fatty acid esters, or fatty esters), terminal olefins, internal olefins, and ketones.

A “fatty acid derivative composition” as referred to herein is produced by a recombinant host cell and typically comprises a mixture of fatty acid derivative. In some cases, the mixture includes more than one type of product (e.g., fatty acids and fatty alcohols, fatty acids and fatty acid esters or alkanes and olefins). In other cases, the fatty acid derivative compositions may comprise, for example, a mixture of fatty alcohols (or another fatty acid derivative) with various chain lengths and saturation or branching characteristics. In still other cases, the fatty acid derivative composition comprises a mixture of both more than one type of product and products with various chain lengths and saturation or branching characteristics.

As used herein “acyl-CoA” refers to an acyl thioester formed between the carbonyl carbon of alkyl chain and the sulfhydryl group of the 4′-phosphopantethionyl moiety of coenzyme A (CoA), which has the formula R-C(O)S-CoA, where R is any alkyl group having at least 4 carbon atoms.

As used herein “acyl-ACP” refers to an acyl thioester formed between the carbonyl carbon of alkyl chain and the sulfhydryl group of the phosphopantetheinyl moiety of an acyl carrier protein (ACP). The phosphopantetheinyl moiety is post-translationally attached to a conserved serine residue on the ACP by the action of holo-acyl carrier protein synthase (ACPS), a phosphopantetheinyl transferase. In some embodiments an acyl-ACP is an intermediate in the synthesis of fully saturated acyl-ACPs. In other embodiments an acyl-ACP is an intermediate in the synthesis of unsaturated acyl-ACPs. In some embodiments, the carbon chain will have about 5, 6, 7, 8, 9, 10, 11, 12, 13, 14, 15, 16, 17, 18, 19, 20, 21, 22, 23, 24, 25, or 26 carbons. Each of these acyl-ACPs are substrates for enzymes that convert them to fatty acid derivatives.

As used herein, the term “fatty acid biosynthetic pathway” means a biosynthetic pathway that produces fatty acids. The fatty acid biosynthetic pathway includes fatty acid synthases that can be engineered to produce fatty acids, and in some embodiments can be expressed with additional enzymes to produce fatty acids having desired carbon chain characteristics.

As used herein, “fatty aldehyde” means an aldehyde having the formula RCHO characterized by a carbonyl group (C═O). In some embodiments, the fatty aldehyde is any aldehyde made from a fatty acid or fatty acid derivative.

As used herein, “fatty alcohol” means an alcohol having the formula ROH. In some embodiments, the fatty alcohol is any alcohol made from a fatty acid or fatty acid derivative.

In certain embodiments, the R group of a fatty acid, fatty aldehyde, or fatty alcohol is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty acid, fatty aldehyde, or fatty alcohol is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty acid, fatty aldehyde, or fatty alcohol. In certain embodiments, the fatty acid, fatty aldehyde, or fatty alcohol is a C₆, C₈, C₁₀, C₁₂, C₁₄, C₁₅, C₁₆, C₁₇, or C₁₈, fatty acid, fatty aldehyde, or fatty alcohol.

The R group of a fatty acid, fatty aldehyde, or fatty alcohol can be a straight chain or a

branched chain. Branched chains may have more than one point of branching and may include cyclic branches. In some embodiments, the branched fatty acid, branched fatty aldehyde, or branched fatty alcohol is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ branched fatty acid, branched fatty aldehyde, or branched fatty alcohol. In particular embodiments, the branched fatty acid, branched fatty aldehyde, or branched fatty alcohol is a C₆, C₈, C₁₀, C₁₂, C₁₄, C₁₅, C₁₆, C₁₇, or C₁₈, branched fatty acid, branched fatty aldehyde, or branched fatty alcohol. In certain embodiments, the hydroxyl group of the branched fatty acid, branched fatty aldehyde, or branched fatty alcohol is in the primary (C₁) position.

In certain embodiments, the branched fatty acid, branched fatty aldehyde, or branched fatty alcohol is an iso-fatty acid, iso-fatty aldehyde, or iso-fatty alcohol, or an antesio-fatty acid, an anteiso-fatty aldehyde, or anteiso-fatty alcohol. In exemplary embodiments, the branched fatty acid, branched fatty aldehyde, or branched fatty alcohol is selected from iso-C_{7:0}, iso-C_{8:0}, iso-C_{9:0}, iso-C_{10:0}, iso-C_{11:0}, iso-C_{12:0}, iso-C_{13:0}, iso-C_{14:0}, iso-C_{15:0}, iso-C_{16:0}, iso-C_{17:0}, iso-C_{18:0}, iso-C_{19:0}, anteiso-C_{7:0}, anteiso-C_{8:0}, anteiso-C_{9:0}, anteiso-C_{10:0}, anteiso-C_{11:0}, anteiso-C_{12:0}, anteiso-C_{13:0}, anteiso-C_{14:0}, anteiso-C_{15:0}, anteiso-C_{16:0}, anteiso-C_{17:0}, anteiso-C_{18:0}, and anteiso-C_{19:0} branched fatty acid, branched fatty aldehyde or branched fatty alcohol.

The R group of a branched or unbranched fatty acid, branched or unbranched fatty aldehyde, or branched or unbranched fatty alcohol can be saturated or unsaturated. If unsaturated, the R group can have one or more than one point of unsaturation. In some embodiments, the unsaturated fatty acid, unsaturated fatty aldehyde, or unsaturated fatty alcohol is a monounsaturated fatty acid, monounsaturated fatty aldehyde, or monounsaturated fatty alcohol. In certain embodiments, the unsaturated fatty acid, unsaturated fatty aldehyde, or unsaturated fatty alcohol is a C6:1, C7:1, C8:1, C9:1, C10:1, C11:1, C12:1, C13:1, C14:1, C15:1,C16:1, C17:1, C18:1, C19:1, C20:1, C21:1, C22:1, C23:1, C24:1, C25:1, or a C26:1 unsaturated fatty acid, unsaturated fatty aldehyde, or unsaturated fatty alcohol. In certain preferred embodiments, the unsaturated fatty acid, unsaturated fatty aldehyde, or unsaturated fatty alcohol is C10:1, C12:1, C14:1, C16:1, or C18:1. In yet other embodiments, the unsaturated fatty acid, unsaturated fatty aldehyde, or unsaturated fatty alcohol is unsaturated at the omega-7 position. In certain embodiments, the unsaturated fatty acid, unsaturated fatty aldehyde, or unsaturated fatty alcohol comprises a cis double bond.

As used herein, the term “alkane” means saturated hydrocarbons or compounds that consist only of carbon (C) and hydrogen (H), wherein these atoms are linked together by single bonds (i.e., they are saturated compounds).

The terms “olefin” and “alkene” are used interchangeably herein, and refer to hydrocarbons containing at least one carbon-to-carbon double bond (i.e., they are unsaturated compounds).

The terms “terminal olefin,” “α-olefin”, “terminal alkene” and “1-alkene” are used interchangeably herein with reference to α-olefins or alkenes with a chemical formula CₓH2ₓ, distinguished from other olefins with a similar molecular formula by linearity of the hydrocarbon chain and the position of the double bond at the primary or alpha position.

As used herein, the term “fatty ester” may be used in reference to an ester. In a preferred embodiment, a fatty ester is any ester made from a fatty acid, for example a fatty acid ester. In some embodiments, a fatty ester contains an A side and a B side. As used herein, an “A side” of an ester refers to the carbon chain attached to the carboxylate oxygen of the ester. As used herein, a “B side” of an ester refers to the carbon chain comprising the parent carboxylate of the ester. In embodiments where the fatty ester is derived from the fatty acid biosynthetic pathway, the A side is contributed by an alcohol, and the B side is contributed by a fatty acid.

Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. When the fatty ester is a fatty acid methyl ester, the A side of the ester is 1 carbon in length. When the fatty ester is a fatty acid ethyl ester, the A side of the ester is 2 carbons in length. The B side of the ester can be at least about 4, 6, 8, 10, 12, 14, 16, 18, 20, 22, 24, or 26 carbons in length. The A side and/or the B side can be straight or branched chain. The branched chains can have one or more points of branching. In addition, the branched chains can include cyclic branches. Furthermore, the A side and/or B side can be saturated or unsaturated. If unsaturated, the A side and/or B side can have one or more points of unsaturation.

In some embodiments, the fatty acid ester is a fatty acid methyl ester (FAME) or a fatty acid ethyl ester (FAEE). In certain embodiments, the FAME is a beta-hydroxy (B-OH) FAME. In one embodiment, the fatty ester is produced biosynthetically. In this embodiment, first the fatty acid is “activated.” Non-limiting examples of “activated” fatty acids are acyl-CoA, acyl ACP, and acyl phosphate. Acyl-CoA can be a direct product of fatty acid biosynthesis or degradation. In addition, acyl-CoA can be synthesized from a free fatty acid, a CoA, and an adenosine nucleotide triphosphate (ATP). An example of an enzyme which produces acyl-CoA is acyl-CoA synthase.

After a fatty acid is activated, it can be readily transferred to a recipient nucleophile. Exemplary nucleophiles are alcohols, thiols, or phosphates.

In one embodiment, the fatty ester is a wax. The wax can be derived from a long chain alcohol and a long chain fatty acid. In another embodiment, the fatty ester is a fatty acid thioester, for example, fatty acyl Coenzyme A (CoA). In other embodiments, the fatty ester is a fatty acyl pantothenate, an acyl carrier protein (ACP), or a fatty phosphate ester.

As used herein “acyl CoA” refers to an acyl thioester formed between the carbonyl carbon of alkyl chain and the sulfydryl group of the 4′-phosphopantethionyl moiety of coenzyme A (CoA), which has the formula R-C(O)S-CoA, where R is any alkyl group having at least 4 carbon atoms. In some instances an acyl CoA will be an intermediate in the synthesis of fully saturated acyl CoAs, including, but not limited to 3-keto-acyl CoA, a 3-hydroxy acyl CoA, a delta-2-trans-enoyl-CoA, or an alkyl acyl CoA. In some embodiments, the carbon chain will have about 5, 6, 7, 8, 9, 10, 11, 12, 13, 14, 15, 16, 17, 18, 19, 20, 21, 22, 23, 24, 25, or 26 carbons. In other embodiments the acyl CoA will be branched. In one embodiment the branched acyl CoA is an isoacyl CoA, in another it is an anti-isoacyl CoA. Each of these “acyl CoAs” are substrates for enzymes that convert them to fatty acid derivatives such as those described herein.

The terms “altered level of expression” and “modified level of expression” are used interchangeably and mean that a polynucleotide, polypeptide, or hydrocarbon is present in a different concentration in an engineered host cell as compared to its concentration in a corresponding wild-type cell under the same conditions.

“Polynucleotide” refers to a polymer of DNA or RNA, which can be single-stranded or double-stranded and which can contain non-natural or altered nucleotides. The terms “polynucleotide,” “nucleic acid,” and “nucleic acid molecule” are used herein interchangeably to refer to a polymeric form of nucleotides of any length, either ribonucleotides (RNA) or deoxyribonucleotides (DNA). These terms refer to the primary structure of the molecule, and thus include double-and single-stranded DNA, and double-and single-stranded RNA. The terms include, as equivalents, analogs of either RNA or DNA made from nucleotide analogs and modified polynucleotides such as, though not limited to methylated and/or capped polynucleotides. The polynucleotide can be in any form, including but not limited to plasmid, viral, chromosomal, EST, cDNA, mRNA, and rRNA.

The term “nucleotide” as used herein refers to a monomeric unit of a polynucleotide that consists of a heterocyclic base, a sugar, and one or more phosphate groups. The naturally occurring bases (guanine, (G), adenine, (A), cytosine, (C), thymine, (T), and uracil (U)) are typically derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleic acids are typically linked via phosphate bonds to form nucleic acids or polynucleotides, though many other linkages are known in the art (e.g., phosphorothioates, boranophosphates, and the like).

Polynucleotides described herein may comprise degenerate nucleotides which are defined according to the IUPAC code for nucleotide degeneracy wherein B is C, G, or T; D is A, G, or T; H is A, C, or T; K is G or T; M is A or C; N is A, C, G, or T; R is A or G; S is C or G; V is A, C, or G; W is A or T; and Y is C or T.

The terms “polypeptide” and “protein” refer to a polymer of amino acid residues. The term “recombinant polypeptide” refers to a polypeptide that is produced by recombinant DNA techniques, wherein generally DNA encoding the expressed protein or RNA is inserted into a suitable expression vector that is in turn used to transform a host cell to produce the polypeptide or RNA.

In some embodiments, the polypeptide, polynucleotide, or hydrocarbon having an altered or modified level of expression is “overexpressed” or has an “increased level of expression.” As used herein, “overexpress” and “increasing the level of expression” mean to express or cause to be expressed a polynucleotide, polypeptide, or hydrocarbon in a cell at a greater concentration than is normally expressed in a corresponding wild-type cell under the same conditions. For example, a polypeptide can be “overexpressed” in an engineered host cell when the polypeptide is present in a greater concentration in the engineered host cell as compared to its concentration in a non-engineered host cell of the same species under the same conditions.

In other embodiments, the polypeptide, polynucleotide, or hydrocarbon having an altered level of expression is “attenuated” or has a “decreased level of expression.” As used herein, “attenuate” and “decreasing the level of expression” mean to express or cause to be expressed a polynucleotide, polypeptide, or hydrocarbon in a cell at a lesser concentration than is normally expressed in a corresponding wild-type cell under the same conditions.

The degree of overexpression or attenuation can be 1.5-fold or more, e.g., 2-fold or more, 3-fold or more, 5-fold or more, 10-fold or more, or 15-fold or more. Alternatively, or in addition, the degree of overexpression or attenuation can be 500-fold or less, e.g., 100-fold or less, 50-fold or less, 25-fold or less, or 20-fold or less. Thus, the degree of overexpression or attenuation can be bounded by any two of the above endpoints. For example, the degree of overexpression or attenuation can be 1.5-500-fold, 2-50-fold, 10-25-fold, or 15-20-fold.

In some embodiments, a polypeptide described herein has “increased level of activity.” By “increased level of activity” is meant that a polypeptide has a higher level of biochemical or biological function (e.g., DNA binding or enzymatic activity) in an engineered host cell as compared to its level of biochemical and/or biological function in a corresponding wild-type host cell under the same conditions. The degree of enhanced activity can be about 10% or more, about 20% or more, about 50% or more, about 75% or more, about 100% or more, about 200% or more, about 500% or more, about 1000% or more, or any range therein.

A polynucleotide or polypeptide can be attenuated using methods known in the art. In some embodiments, the expression of a gene or polypeptide encoded by the gene is attenuated by mutating the regulatory polynucleotide sequences which control expression of the gene. In other embodiments, the expression of a gene or polypeptide encoded by the gene is attenuated by overexpressing a repressor protein, or by providing an exogenous regulatory element that activates a repressor protein. In still yet other embodiments, DNA- or RNA-based gene silencing methods are used to attenuate the expression of a gene or polynucleotide. In some embodiments, the expression of a gene or polypeptide is completely attenuated, e.g., by deleting all or a portion of the polynucleotide sequence of a gene.

A polynucleotide or polypeptide can be overexpressed using methods known in the art. In some embodiments, overexpression of a polypeptide is achieved by the use of an exogenous regulatory element. The term “exogenous regulatory element” generally refers to a regulatory element originating outside of the host cell. However, in certain embodiments, the term “exogenous regulatory element” can refer to a regulatory element derived from the host cell whose function is replicated or usurped for the purpose of controlling the expression of an endogenous polypeptide. For example, if the host cell is an E. coli cell, and the FadR polypeptide is a encoded by an endogenous fadR gene, then expression of the endogenous fadR can be controlled by a promoter derived from another E. coli gene.

In some embodiments, the exogenous regulatory element is a chemical compound, such as a small molecule. As used herein, the term “small molecule” refers to a substance or compound having a molecular weight of less than about 1,000 g/mol.

In some embodiments, the exogenous regulatory element which controls the expression of an endogenous fadR gene is an expression control sequence which is operably linked to the endogenous fadR gene by recombinant integration into the genome of the host cell. In certain embodiments, the expression control sequence is integrated into a host cell chromosome by homologous recombination using methods known in the art (e.g., Datsenko et al., Proc. Natl. Acad. Sci. U.S.A., 97(12): 6640-6645 (2000)).

Expression control sequences are known in the art and include, for example, promoters, enhancers, polyadenylation signals, transcription terminators, internal ribosome entry sites (IRES), ribosome binding sites (RBS) and the like, that provide for the expression of the polynucleotide sequence in a host cell. Expression control sequences interact specifically with cellular proteins involved in transcription (Maniatis et al., Science, 236:1237-1245 (1987)). Exemplary expression control sequences are described in, for example, Goeddel, Gene Expression Technology: Methods in Enzymology, Vol. 185, Academic Press, San Diego, Calif. (1990).

In the methods of the invention, an expression control sequence is operably linked to a polynucleotide sequence. By “operably linked” is meant that a polynucleotide sequence and an expression control sequence(s) are connected in such a way as to permit gene expression when the appropriate molecules (e.g., transcriptional activator proteins) are bound to the expression control sequence(s). Operably linked promoters are located upstream of the selected polynucleotide sequence in terms of the direction of transcription and translation. Operably linked enhancers can be located upstream, within, or downstream of the selected polynucleotide.

In some embodiments, the polynucleotide sequence is provided to the host cell by way of a recombinant vector, which comprises a promoter operably linked to the polynucleotide sequence. In certain embodiments, the promoter is a developmentally-regulated, an organelle-specific, a tissue-specific, an inducible, a constitutive, or a cell-specific promoter.

As used herein, the term “vector” refers to a nucleic acid molecule capable of transporting another nucleic acid, i.e., a polynucleotide sequence, to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids,” which refer generally to circular double stranded DNA loops that, in their vector form, are not bound to the chromosome. The terms “plasmid” and “vector” are used interchangeably herein, inasmuch as a plasmid is the most commonly used form of vector. However, also included are such other forms of expression vectors that serve equivalent functions and that become known in the art subsequently hereto.

The term “regulatory sequences” as used herein typically refers to a sequence of bases in DNA, operably-linked to DNA sequences encoding a protein that ultimately controls the expression of the protein. Examples of regulatory sequences include, but are not limited to, RNA promoter sequences, transcription factor binding sequences, transcription termination sequences, modulators of transcription (such as enhancer elements), nucleotide sequences that affect RNA stability, and translational regulatory sequences (such as, ribosome binding sites (e.g., Shine-Dalgarno sequences in prokaryotes or Kozak sequences in eukaryotes), initiation codons, termination codons).

As used herein, the phrase “the expression of said nucleotide sequence is modified relative to the wild type nucleotide sequence,” means an increase or decrease in the level of expression and/or activity of an endogenous nucleotide sequence or the expression and/or activity of a heterologous or non-native polypeptide-encoding nucleotide sequence.

As used herein, the term “express” with respect to a polynucleotide is to cause it to function. A polynucleotide which encodes a polypeptide (or protein) will, when expressed, be transcribed and translated to produce that polypeptide (or protein). As used herein, the term “overexpress” means to express or cause to be expressed a polynucleotide or polypeptide in a cell at a greater concentration than is normally expressed in a corresponding wild-type cell under the same conditions.

In some embodiments, the recombinant vector comprises at least one sequence selected from the group consisting of (a) an expression control sequence operatively coupled to the polynucleotide sequence; (b) a selection marker operatively coupled to the polynucleotide sequence; (c) a marker sequence operatively coupled to the polynucleotide sequence; (d) a purification moiety operatively coupled to the polynucleotide sequence; (e) a secretion sequence operatively coupled to the polynucleotide sequence; and (f) a targeting sequence operatively coupled to the polynucleotide sequence.

The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides, encoded by the polynucleotide sequences as described herein.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino-or carboxy-terminus of the recombinant polypeptide. Such fusion vectors typically serve one or more of the following three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech, Inc., Piscataway, NJ; Smith et al., Gene, 67:31-40 (1988)), pMAL (New England Biolabs, Beverly, MA), and pRITS (Pharmacia Biotech, Inc., Piscataway, N.J.), which fuse glutathione S-transferase (GST), maltose E binding protein, or protein A, respectively, to the target recombinant polypeptide.

Suitable expression systems for both prokaryotic and eukaryotic cells are well known in the art; see, e.g., Sambrook et al., “Molecular Cloning: A Laboratory Manual,” second edition, Cold Spring Harbor Laboratory (1989). Examples of inducible, non-fusion E. coli expression vectors include pTrc (Amann et al., Gene, 69:301-315 (1988)) and PET 11d (Studier et al., Gene Expression Technology: Methods in Enzymology 185, Academic Press, San Diego, CA, pp. 60-89 (1990)). In certain embodiments, a polynucleotide sequence of the invention is operably linked to a promoter derived from bacteriophage T5. Examples of vectors for expression in yeast include pYepSec1 (Baldari et al., EMBO J., 6:229-234 (1987)), pMFa (Kurjan et al., Cell, 30:933-943 (1982)), pJRY88 (Schultz et al., Gene, 54:113-123 (1987)), pYES2 (Invitrogen Corp., San Diego, CA), and picZ (Invitrogen Corp., San Diego, CA). Baculovirus vectors available for expression of proteins in cultured insect cells (e.g., Sf9 cells) include, for example, the pAc series (Smith et al., Mol. Cell Biol., 3:2156-2165 (1983)) and the pVL series (Lucklow et al., Virology, 170:31-39 (1989)). Examples of mammalian expression vectors include pCDM8 (Seed, Nature, 329:840 (1987)) and pMT2PC (Kaufinan et al., EMBO J., 6:187-195 (1987)).

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to an antibiotic) can be introduced into the host cells along with the gene of interest. Selectable markers include those that confer resistance to drugs such as, but not limited to, ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transformed with the introduced nucleic acid can be identified by growth in the presence of an appropriate selection drug.

Similarly, for stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to an antibiotic) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by growth in the presence of an appropriate selection drug.

In some embodiments, the FadR polypeptide has the amino acid sequence of SEQ ID NO: 1.

In other embodiments, the FadR polypeptide is encoded by a fadR gene obtained from microorganisms of the genera Escherichia, Salmonella, Citrobacter, Enterobacter, Klebsiella, Cronobacter, Yersinia, Serratia, Erwinia, Pectobacterium, Photorhabdus, Edwardsiella, Shewanella, or Vibrio.

In other embodiments, the FadR polypeptide is a homologue of FadR having an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 1.

The identity of a FadR polypeptide having at least 80% identity to the amino acid sequence of SEQ ID NO: 1 is not particularly limited, and one of ordinary skill in the art can readily identify homologues of E. coli MG1655 derived-FadR using the methods described herein as well as methods known in the art.

As used herein, the terms “homolog,” and “homologous” refer to a polynucleotide or a polypeptide comprising a sequence that is at least about 50% identical to the corresponding polynucleotide or polypeptide sequence. Preferably homologous polynucleotides or polypeptides have polynucleotide sequences or amino acid sequences that have at least about 80%, 81%, 82%, 83%, 84%, 85%, 86%, 87%, 88%, 89%, 90%, 91%, 2%, 93%, 94%, 95%, 96%, 97%, 98% or at least about 99% homology to the corresponding amino acid sequence or polynucleotide sequence. As used herein the terms sequence “homology” and sequence “identity” are used interchangeably.

Briefly, calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes. The amino acid residues or nucleotides at corresponding amino acid positions or nucleotide positions of the first and second sequences are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences.

The comparison of sequences and determination of percent homology between two sequences can be accomplished using a mathematical algorithm, such as BLAST (Altschul et al., J. Mol. Biol., 215(3): 403-410 (1990)). The percent homology between two amino acid sequences also can be determined using the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3,4, 5, or 6 (Needleman and Wunsch, J. Mol. Biol., 48:444-453 (1970)). The percent homology between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial homology calculations and adjust the algorithm parameters accordingly. A preferred set of parameters (and the one that should be used if a practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5. Additional methods of sequence alignment are known in the biotechnology arts (see, e.g., Rosenberg, BMC Bioinformatics, 6:278 (2005); Altschul et al., FEBS J., 272(20): 5101-5109 (2005)).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either method can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2.×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions unless otherwise specified.

In some embodiments, the polypeptide is a fragment of any of the polypeptides described herein. The term “fragment” refers to a shorter portion of a full-length polypeptide or protein ranging in size from four amino acid residues to the entire amino acid sequence minus one amino acid residue. In certain embodiments of the invention, a fragment refers to the entire amino acid sequence of a domain of a polypeptide or protein (e.g., a substrate binding domain or a catalytic domain).

An “endogenous” polypeptide refers to a polypeptide encoded by the genome of the parental microbial cell (also termed “host cell”) from which the recombinant cell is engineered (or “derived”).

An “exogenous” polypeptide refers to a polypeptide which is not encoded by the genome of the parental microbial cell. A variant (i.e., mutant) polypeptide is an example of an exogenous polypeptide.

The term “heterologous” as used herein typically refers to a nucleotide sequence or a protein not naturally present in an organism. For example, a polynucleotide sequence endogenous to a plant can be introduced into a host cell by recombinant methods, and the plant polynucleotide is then a heterologous polynucleotide in a recombinant host cell.

In some embodiments, the polypeptide is a mutant or a variant of any of the polypeptides described herein. The terms “mutant” and “variant” as used herein refer to a polypeptide having an amino acid sequence that differs from a wild-type polypeptide by at least one amino acid. For example, the mutant can comprise one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. In some embodiments, the mutant polypeptide has about 1, 2, 3, 4, 5, 6, 7, 8, 9, 10, 15, 20, 30, 40, 50, 60, 70, 80, 90, 100, or more amino acid substitutions, additions, insertions, or deletions.

As used herein, the term “mutagenesis” refers to a process by which the genetic information of an organism is changed in a stable manner. Mutagenesis of a protein coding nucleic acid sequence produces a mutant protein. Mutagenesis also refers to changes in non-coding nucleic acid sequences that result in modified protein activity.

As used herein, the term “gene” refers to nucleic acid sequences encoding either an RNA product or a protein product, as well as operably-linked nucleic acid sequences affecting the expression of the RNA or protein (e.g., such sequences include but are not limited to promoter or enhancer sequences) or operably-linked nucleic acid sequences encoding sequences that affect the expression of the RNA or protein (e.g., such sequences include but are not limited to ribosome binding sites or translational control sequences).

In certain embodiments, the FadR polypeptide comprises a mutation at an amino acid residue corresponding to amino acid 219 of SEQ ID NO: 1. In certain embodiments, the mutation results in a substitution of the amino acid residue corresponding to amino acid 219 of SEQ ID NO: 1 with an asparagine residue. The FadR(S219N) mutation has been previously described (Raman et al., J. Biol. Chem., 270:1092-1097 (1995)).

Preferred fragments or mutants of a polypeptide retain some or all of the biological function (e.g., enzymatic activity) of the corresponding wild-type polypeptide. In some embodiments, the fragment or mutant retains at least 75%, at least 80%, at least 90%, at least 95%, or at least 98% or more of the biological function of the corresponding wild-type polypeptide. In other embodiments, the fragment or mutant retains about 100% of the biological function of the corresponding wild-type polypeptide. Guidance in determining which amino acid residues may be substituted, inserted, or deleted without affecting biological activity may be found using computer programs well known in the art, for example, LASERGENE™ software (DNASTAR, Inc., Madison, WI).

In yet other embodiments, a fragment or mutant exhibits increased biological function as compared to a corresponding wild-type polypeptide. For example, a fragment or mutant may display at least a 10%, at least a 25%, at least a 50%, at least a 75%, or at least a 90% improvement in enzymatic activity as compared to the corresponding wild-type polypeptide. In other embodiments, the fragment or mutant displays at least 100% (e.g., at least 200%, or at least 500%) improvement in enzymatic activity as compared to the corresponding wild-type polypeptide.

It is understood that the polypeptides described herein may have additional conservative or non-essential amino acid substitutions, which do not have a substantial effect on the polypeptide function. Whether or not a particular substitution will be tolerated (i.e., will not adversely affect desired biological function, such as DNA binding or enzyme activity) can be determined as described in Bowie et al. (Science, 247:1306-1310 (1990)).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

Variants can be naturally occurring or created in vitro. In particular, such variants can be created using genetic engineering techniques, such as site directed mutagenesis, random chemical mutagenesis, Exonuclease III deletion procedures, or standard cloning techniques. Alternatively, such variants, fragments, analogs, or derivatives can be created using chemical synthesis or modification procedures.

Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be prepared by using random and site-directed mutagenesis (see, e.g., Arnold, Curr. Opin. Biotech., 4:450-455 (1993)). Random mutagenesis can be achieved using error prone PCR (see, e.g., Leung et al., Technique, 1:11-15 (1989); and Caldwell et al., PCR Methods Applic., 2:28-33 (1992)). Site-directed mutagenesis can be achieved using oligonucleotide-directed mutagenesis to generate site-specific mutations in any cloned DNA of interest (see, e.g., Reidhaar-Olson et al., Science, 241:53-57 (1988)). Other methods for generating variants include, e.g., assembly PCR (see, e.g., U.S. Pat. No. 5,965,408), sexual PCR mutagenesis (see, e.g., Stemmer, Proc. Natl. Acad. Sci., U.S.A., 91:10747-10751 (1994) and U.S. Pat. Nos. 5,965,408 and 5,939,250), recursive ensemble mutagenesis (see, e.g., Arkin et al., Proc. Natl. Acad. Sci., U.S.A., 89:7811-7815 (1992)), and exponential ensemble mutagenesis (see, e.g., Delegrave et al., Biotech. Res, 11:1548-1552 (1993).

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence (e.g., a polynucleotide sequence encoding a PPTase) in one of these strains will eventually generate random mutations within the DNA. Mutator strains suitable for use for in vivo mutagenesis are described in, for example, International Patent Application Publication No. WO 1991/016427.

Variants can also be generated using cassette mutagenesis. In cassette mutagenesis, a small region of a double-stranded DNA molecule is replaced with a synthetic oligonucleotide “cassette” that differs from the native sequence. The oligonucleotide often contains a completely and/or partially randomized native sequence.

As used herein, a “host cell” is a cell used to produce a product described herein (e.g., a fatty aldehyde or a fatty alcohol). In any of the aspects of the invention described herein, the host cell can be selected from the group consisting of a mammalian cell, plant cell, insect cell, fungus cell (e.g., a filamentous fungus cell or a yeast cell), and bacterial cell. A host cell is referred to as an “engineered host cell” or a “recombinant host cell” if the expression of one or more polynucleotides or polypeptides in the host cell are altered or modified as compared to their expression in a corresponding wild-type host cell under the same conditions.

In some embodiments, the host cell is a Gram-positive bacterial cell. In other embodiments, the host cell is a Gram-negative bacterial cell.

In some embodiments, the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In other embodiments, the host cell is a Bacillus lentus cell, a Bacillus brevis cell, a Bacillus stearothermophilus cell, a Bacillus lichen formis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell.

In yet other embodiments, the host cell is an Actinomycetes cell.

In some embodiments, the host cell is a Saccharomyces cerevisiae cell. In some embodiments, the host cell is a Saccharomyces cerevisiae cell.

In still other embodiments, the host cell is a CHO cell, a COS cell, a VERO cell, a BHK cell, a HeLa cell, a Cv1 cell, an MDCK cell, a 293 cell, a 3T3 cell, or a PC12 cell.

In other embodiments, the host cell is a cell from a eukaryotic plant, algae, cyanobacterium, green-sulfur bacterium, green non-sulfur bacterium, purple sulfur bacterium, purple non-sulfur bacterium, extremophile, yeast, fungus, an engineered organism thereof, or a synthetic organism. In some embodiments, the host cell is light-dependent or fixes carbon. In some embodiments, the host cell is light-dependent or fixes carbon. In some embodiments, the host cell has autotrophic activity. In some embodiments, the host cell has photoautotrophic activity, such as in the presence of light. In some embodiments, the host cell is heterotrophic or mixotrophic in the absence of light. In certain embodiments, the host cell is a cell from Avabidopsis thaliana, Panicum virgatum, Miscanthus giganteus, Zea mays, Botryococcuse braunii, Chlamydomonas reinhardtii, Dunaliela salina, Synechococcus Sp. PCC 7002, Synechococcus Sp. PCC 7942, Synechocystis Sp. PCC 6803, Thermosynechococcus elongates BP-1, Chlorobium tepidum, Chlorojlexus auranticus, Chromatiumm vinosum, Rhodospirillum rubrum, Rhodobacter capsulatus, Rhodopseudomonas palusris, Clostridium ljungdahlii, Clostridiuthermocellum, Penicillium chrysogenum, Pichia pastoris, Saccharomyces cerevisiae, Schizosaccharomyces pombe, Pseudomonasjluorescens, or Zymomonas mobilis.

In certain preferred embodiments, the host cell is an E. coli cell. In some embodiments, the E. coli cell is a strain B, a strain C, a strain K, or a strain W E. coli cell.

In other embodiments, the host cell is a Pantoea citrea cell.

As used herein, the term “conditions permissive for the production” means any conditions that allow a host cell to produce a desired product, such as a fatty acid or a fatty acid derivative. Similarly, the term “conditions in which the polynucleotide sequence of a vector is expressed” means any conditions that allow a host cell to synthesize a polypeptide. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source that can be metabolized by a host cell directly. In addition, enzymes can be used in the medium to facilitate the mobilization (e.g., the depolymerization of starch or cellulose to fermentable sugars) and subsequent metabolism of the carbon source.

As used herein, the phrase “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, and turanose; cellulosic material and variants such as methyl cellulose and sodium carboxymethyl cellulose; saturated or unsaturated fatty acid esters, succinate, lactate, and acetate; alcohols, such as ethanol, methanol, and glycerol, or mixtures thereof. The carbon source can also be a product of photosynthesis, such as glucose. In certain preferred embodiments, the carbon source is biomass. In other preferred embodiments, the carbon source is glucose. In still other preferred embodiments, the carbon source is sucrose.

As used herein, the term “biomass” refers to any biological material from which a carbon source is derived. In some embodiments, a biomass is processed into a carbon source, which is suitable for bioconversion. In other embodiments, the biomass does not require further processing into a carbon source. The carbon source can be converted into a biofuel. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers. The term “biomass” also can refer to sources of carbon, such as carbohydrates (e.g., monosaccharides, disaccharides, or polysaccharides).

As used herein, the term “clone” typically refers to a cell or group of cells descended from and essentially genetically identical to a single common ancestor, for example, the bacteria of a cloned bacterial colony arose from a single bacterial cell.

As used herein, the term “culture” typical refers to a liquid media comprising viable cells. In one embodiment, a culture comprises cells reproducing in a predetermined culture media under controlled conditions, for example, a culture of recombinant host cells grown in liquid media comprising a selected carbon source and nitrogen.

“Culturing” or “cultivation” refers to growing a population of recombinant host cells under suitable conditions in a liquid or solid medium. In particular embodiments, culturing refers to the fermentative bioconversion of a substrate to an end-product. Culturing media are well known and individual components of such culture media are available from commercial sources, e.g., under the Difco™ and BBL™ trademarks. In one non-limiting example, the aqueous nutrient medium is a “rich medium” comprising complex sources of nitrogen, salts, and carbon, such as YP medium, comprising 10 g/L of peptone and 10 g/L yeast extract of such a medium.

To determine if conditions are sufficient to allow production of a product or expression of a polypeptide, a host cell can be cultured, for example, for about 4, 8, 12, 24, 36, 48, 72, or more hours. During and/or after culturing, samples can be obtained and analyzed to determine if the conditions allow production or expression. For example, the host cells in the sample or the medium in which the host cells were grown can be tested for the presence of a desired product. When testing for the presence of a fatty acid or fatty acid derivative, assays, such as, but not limited to, mass spectrometry (MS), thin layer chromatography (TLC), high-performance liquid chromatography (HPLC), liquid chromatography (LC), GC coupled with a flame ionization detector (FID), GC-MS, and liquid chromatography-mass spectrometry (LC-MS) can be used. When testing for the expression of a polypeptide, techniques such as, but not limited to, Western blotting and dot blotting may be used.

In the compositions and methods of the invention, the production and isolation of fatty acids and fatty acid derivatives can be enhanced by optimizing fermentation conditions. In some embodiments, fermentation conditions are optimized to increase the percentage of the carbon source that is converted to hydrocarbon products. During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science 311:1113 (2006); Venturi, FEMS Microbiol. Rev., 30:274-291 (2006); and Reading et al., FEMS Microbiol. Lett., 254:1-11 (2006)) can be used to activate checkpoint genes, such as p53, p21, or other checkpoint genes.

Genes that can be activated to stop cell replication and growth in E. coli include umuDC genes. The overexpression of umuDC genes stops the progression from stationary phase to exponential growth (Murli et al., J. Bacteriol., 182:1127-1135 (2000)). UmuC is a DNA polymerase that can carry out translesion synthesis over non-coding lesions which commonly result from ultraviolet (UV) and chemical mutagenesis. The umuDC gene products are involved in the process of translesion synthesis and also serve as a DNA sequence damage checkpoint. The umuDC gene products include UmuC, UmuD, umuD′, UmuD′₂C, UmuD′₂, and UmuD₂. Simultaneously, product-producing genes can be activated, thereby minimizing the need for replication and maintenance pathways to be used while a fatty aldehyde or fatty alcohol is being made. Host cells can also be engineered to express umuC and umuD from E. coli in pBAD24 under the prpBCDE promoter system through de novo synthesis of this gene with the appropriate end-product production genes.

The host cell can be additionally engineered to express a recombinant cellulosome, which can allow the host cell to use cellulosic material as a carbon source. Exemplary cellulosomes suitable for use in the methods of the invention include, e.g, the cellulosomes described in International Patent Application Publication WO 2008/100251. The host cell also can be engineered to assimilate carbon efficiently and use cellulosic materials as carbon sources according to methods described in U.S. Pat. Nos. 5,000,000; 5,028,539; 5,424,202; 5,482,846; and 5,602,030. In addition, the host cell can be engineered to express an invertase so that sucrose can be used as a carbon source.

In some embodiments of the fermentation methods of the invention, the fermentation chamber encloses a fermentation that is undergoing a continuous reduction, thereby creating a stable reductive environment. The electron balance can be maintained by the release of carbon dioxide (in gaseous form). Efforts to augment the NAD/H and NADP/H balance can also facilitate in stabilizing the electron balance. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of fatty aldehydes and fatty alcohols.

For small scale production, the engineered host cells can be grown in batches of, for example, about 100 mL, 500 mL, 1 L, 2 L, 5 L, or 10 L; fermented; and induced to express a desired polynucleotide sequence, such as a polynucleotide sequence encoding a PPTase. For large scale production, the engineered host cells can be grown in batches of about 10 L, 100 L, 1000 L, 10,000 L, 100,000 L, 1,000,000 L or larger; fermented; and induced to express a desired polynucleotide sequence.

The fatty acids and derivatives thereof produced by the methods of invention generally are isolated from the host cell. The term “isolated” as used herein with respect to products, such as fatty acids and derivatives thereof, refers to products that are separated from cellular components, cell culture media, or chemical or synthetic precursors. The fatty acids and derivatives thereof produced by the methods described herein can be relatively immiscible in the fermentation broth, as well as in the cytoplasm. Therefore, the fatty acids and derivatives thereof can collect in an organic phase either intracellularly or extracellularly. The collection of the products in the organic phase can lessen the impact of the fatty acid or fatty acid derivative on cellular function and can allow the host cell to produce more product.

In some embodiments, the fatty acids and fatty acid derivatives produced by the methods of invention are purified. As used herein, the term “purify,” “purified,” or “purification” means the removal or isolation of a molecule from its environment by, for example, isolation or separation. “Substantially purified” molecules are at least about 60% free (e.g., at least about 70% free, at least about 75% free, at least about 85% free, at least about 90% free, at least about 95% free, at least about 97% free, at least about 99% free) from other components with which they are associated. As used herein, these terms also refer to the removal of contaminants from a sample. For example, the removal of contaminants can result in an increase in the percentage of a fatty aldehyde or a fatty alcohol in a sample. For example, when a fatty aldehyde or a fatty alcohol is produced in a host cell, the fatty aldehyde or fatty alcohol can be purified by the removal of host cell proteins. After purification, the percentage of a fatty acid or derivative thereof in the sample is increased.

As used herein, the terms “purify,” “purified,” and “purification” are relative terms which do not require absolute purity. Thus, for example, when a fatty acid or derivative thereof is produced in host cells, a purified fatty acid or derivative thereof is a fatty acid or derivative thereof that is substantially separated from other cellular components (e.g., nucleic acids, polypeptides, lipids, carbohydrates, or other hydrocarbons).

Additionally, a purified fatty acid preparation or a purified fatty acid derivative preparation is a fatty acid preparation or a fatty acid derivative preparation in which the fatty acid or derivative thereof is substantially free from contaminants, such as those that might be present following fermentation. In some embodiments, a fatty acid or derivative thereof is purified when at least about 50% by weight of a sample is composed of the fatty acid or fatty acid derivative. In other embodiments, a fatty acid or derivative thereof is purified when at least about 60%, e.g., at least about 70%, at least about 80%, at least about 85%, at least about 90%, at least about 92% or more by weight of a sample is composed of the fatty acid or derivative thereof. Alternatively, or in addition, a fatty acid or derivative thereof is purified when less than about 100%, e.g., less than about 99%, less than about 98%, less than about 95%, less than about 90%, or less than about 80% by weight of a sample is composed of the fatty acid or derivative thereof. Thus, a purified fatty acid or derivative thereof can have a purity level bounded by any two of the above endpoints. For example, a fatty acid or derivative thereof can be purified when at least about 80%-95%, at least about 85%-99%, or at least about 90%-98% of a sample is composed of the fatty acid or fatty acid derivative.

The fatty acid or derivative thereof may be present in the extracellular environment, or it may be isolated from the extracellular environment of the host cell. In certain embodiments, a fatty acid or derivative thereof is secreted from the host cell. In other embodiments, a fatty acid or derivative thereof is transported into the extracellular environment. In yet other embodiments, the fatty acid or derivative thereof is passively transported into the extracellular environment. A fatty acid or derivative thereof can be isolated from a host cell using methods known in the art, such as those disclosed in International Patent Application Publications WO 2010/042664 and WO 2010/062480.

The methods described herein can result in the production of homogeneous compounds wherein at least about 60%, at least about 70%, at least about 80%, at least about 90%, or at least about 95%, of the fatty acids or fatty acid derivatives produced will have carbon chain lengths that vary by less than 6 carbons, less than 5 carbons, less than 4 carbons, less than 3 carbons, or less than about 2 carbons. Alternatively, or in addition, the methods described herein can result in the production of homogeneous compounds wherein less than about 98%, less than about 95%, less than about 90%, less than about 80%, or less than about 70% of the fatty acids or fatty acid derivatives produced will have carbon chain lengths that vary by less than 6 carbons, less than 5 carbons, less than 4 carbons, less than 3 carbons, or less than about 2 carbons. Thus, the fatty acids or fatty acid derivatives can have a degree of homogeneity bounded by any two of the above endpoints. For example, the fatty acid or fatty acid derivative can have a degree of homogeneity wherein about 70%-95%, about 80%-98%, or about 90%-95% of the fatty acids or fatty acid derivatives produced will have carbon chain lengths that vary by less than 6 carbons, less than 5 carbons, less than 4 carbons, less than 3 carbons, or less than about 2 carbons. These compounds can also be produced with a relatively uniform degree of saturation.

As a result of the methods of the present invention, one or more of the titer, yield, or productivity of the fatty acid or derivative thereof produced by the engineered host cell having an altered level of expression of a FadR polypeptide is increased relative to that of the corresponding wild-type host cell.

The term “titer” refers to the quantity of fatty acid or fatty acid derivative produced per unit volume of host cell culture. In any aspect of the compositions and methods described herein, a fatty acid or a fatty acid derivative such as a terminal olefin, a fatty aldehyde, a fatty alcohol, an alkane, a fatty ester, a ketone or an internal olefins is produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 g/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L, or a range bounded by any two of the foregoing values. In other embodiments, a fatty acid or fatty acid derivative is produced at a titer of more than 2000 mg/L, more than 5000 mg/L, more than 10,000 mg/L, or higher, such as 50 g/L, 70 g/L, 100 g/L, 120 g/L, 150 g/L, or 200 g/L.

As used herein, the “yield of fatty acid derivative produced by a host cell” refers to the efficiency by which an input carbon source is converted to product (i.e., fatty alcohol or fatty aldehyde) in a host cell. Host cells engineered to produce fatty acid derivatives according to the methods of the invention have a yield of at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% or a range bounded by any two of the foregoing values. In other embodiments, a fatty acid derivative or derivatives is produced at a yield of more than 30%, 40%, 50%, 60%, 70%, 80%, 90% or more. Alternatively, or in addition, the yield is about 30% or less, about 27% or less, about 25% or less, or about 22% or less. Thus, the yield can be bounded by any two of the above endpoints. For example, the yield of a fatty acid derivative or derivatives produced by the recombinant host cell according to the methods of the invention can be 5% to 15%, 10% to 25%, 10% to 22%, 15% to 27%, 18% to 22%, 20% to 28%, or 20% to 30%. The yield may refer to a particular fatty acid derivative or a combination of fatty acid derivatives produced by a given recombinant host cell culture.

In one approach, the term “productivity of the fatty acid or derivative thereof produced by a host cell” refers to the quantity of fatty acid or fatty acid derivative produced per unit volume of host cell culture per unit density of host cell culture. In any aspect of the compositions and methods described herein, the productivity of a fatty acid or a fatty acid derivative such as an olefin, a fatty aldehyde, a fatty alcohol, an alkane, a fatty ester, or a ketone produced by an engineered host cells is at least about at least about 3 mg/L/OD₆₀₀, at least about 6 mg/L/OD₆₀₀, at least about 9 mg/L/OD₆₀₀, at least about 12 mg/L/OD₆₀₀, or at least about 15 mg/L/OD₆₀₀. Alternatively, or in addition, the productivity is about 50 mg/L/OD₆₀₀ or less, about 40 mg/L/OD₆₀₀ or less, about 30 mg/L/OD₆₀₀ or less, or about 20 mg/L/OD₆₀₀ or less. Thus, the productivity can be bounded by any two of the above endpoints. For example, the productivity can be about 3 to about 30 mg/L/OD₆₀₀, about 6 to about 20 mg/L/OD₆₀₀, or about 15 to about 30 mg/L/OD₆₀₀.

In another approach, the term “productivity” refers to the quantity of a fatty acid derivative or derivatives produced per unit volume of host cell culture per unit time. In any aspect of the compositions and methods described herein, the productivity of a fatty acid derivative or derivatives produced by a recombinant host cell is at least 100 mg/L/hour, at least 200 mg/L/hour0, at least 300 mg/L/hour, at least 400 mg/L/hour, at least 500 mg/L/hour, at least 600 mg/L/hour, at least 700 mg/L/hour, at least 800 mg/L/hour, at least 900 mg/L/hour, at least 1000 mg/L/hour, at least 1100 mg/L/hour, at least 1200 mg/L/hour, at least 1300 mg/L/hour, at least 1400 mg/L/hour, at least 1500 mg/L/hour, at least 1600 mg/L/hour, at least 1700 mg/L/hour, at least 1800 mg/L/hour, at least 1900 mg/L/hour, at least 2000 mg/L/hour, at least 2100 mg/L/hour, at least 2200 mg/L/hour, at least 2300 mg/L/hour, at least 2400 mg/L/hour, or at least 2500 mg/L/hour. Alternatively, or in addition, the productivity is 2500 mg/L/hour or less, 2000 mg/L/OD600 or less, 1500 mg/L/OD600 or less, 120 mg/L/hour, or less, 1000 mg/L/hour or less, 800 mg/L/hour, or less, or 600 mg/L/hour or less. Thus, the productivity can be bounded by any two of the above endpoints. For example, the productivity can be 3 to 30 mg/L/hour, 6 to 20 mg/L/hour, or 15 to 30 mg/L/hour. For example, the productivity of a fatty acid derivative or derivatives produced by a recombinant host cell according to the methods of the may be from 500 mg/L/hour to 2500 mg/L/hour, or from 700 mg/L/hour to 2000 mg/L/hour. The productivity may refer to a particular fatty acid derivative or a combination of fatty acid derivatives produced by a given recombinant host cell culture.

In the compositions and methods of the invention, the production and isolation of a desired fatty acid or derivative thereof (e.g., acyl-CoA, fatty acids, terminal olefins, fatty aldehydes, fatty alcohols, alkanes, alkenes, wax esters, ketones and internal olefins) can be enhanced by altering the expression of one or more genes involved in the regulation of fatty acid, fatty ester, alkane, alkene, olefin fatty alcohol production, degradation and/or secretion in the engineered host cell.

FadR is known to modulate the expression and/or activity of numerous genes, including fabA, fabB, iclR, fadA, fadB, fadD, fadE, fadI, fadJ, fadL, fadM, uspA, aceA, aceB, and aceK. In some embodiments of the methods described herein, the engineered host cell further comprises an altered level of expression of one or more genes selected from the group consisting of fabA, fabB, iclR, fadA, fadB, fadD, fadE, fadI, fadJ, fadL, fadM, uspA, aceA, aceB, and aceK as compared to the level of expression of the selected gene(s) in a corresponding wild-type host cell. Exemplary accession numbers for polypeptides encoded by the FadR target genes include fabA (NP_415474), fabB (BAA16180), (NP_418442), fadA (YP_026272.1), fadB (NP_418288.1), fadD (AP_002424), fadE (NP_414756.2), fadI (NP_416844.1), fadJ (NP_416843.1), fadL (AAC75404), fadM (NP_414977.1), uspA (AAC76520), aceA (AAC76985.1), aceB (AAC76984.1), and aceK (AAC76986.1).

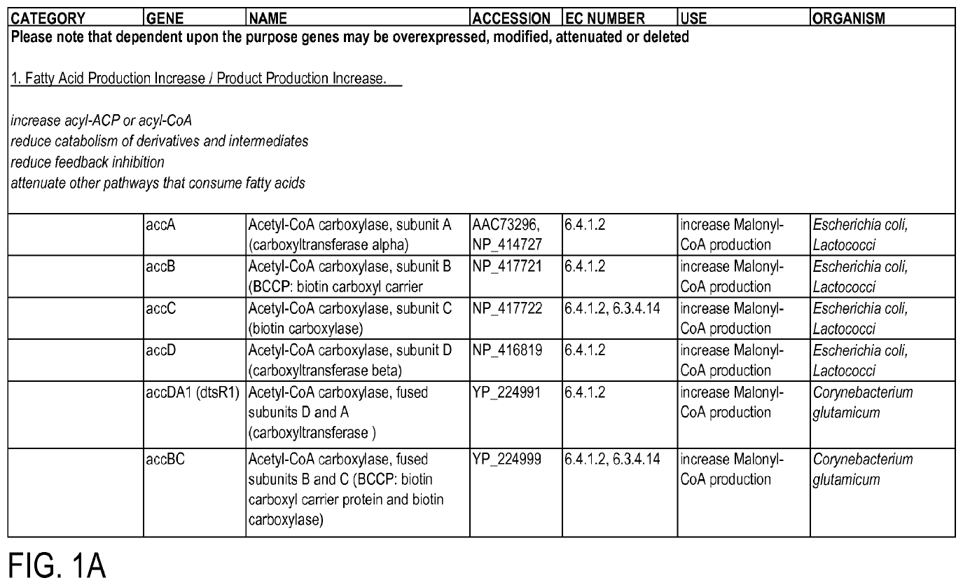

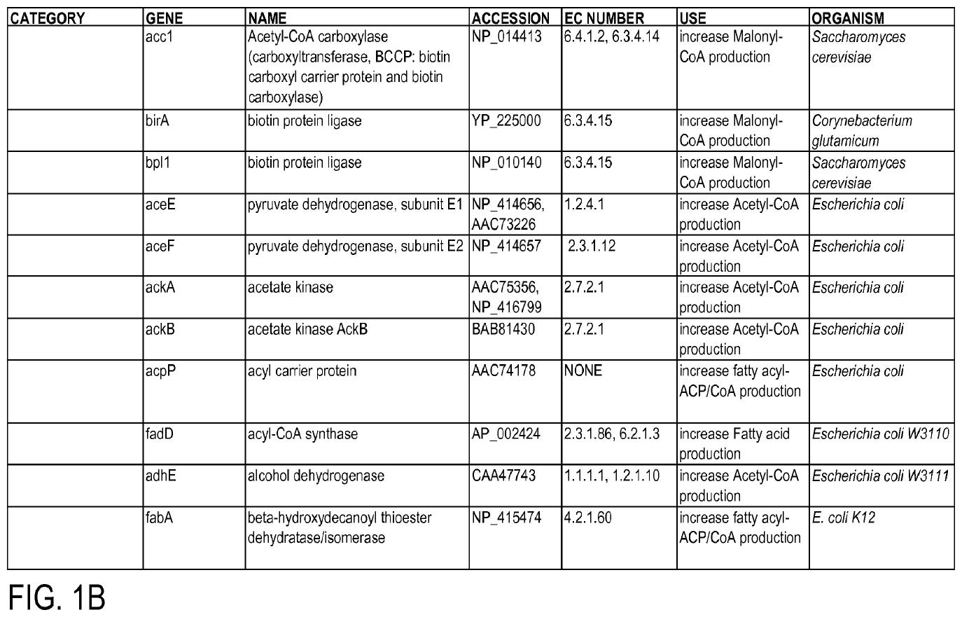

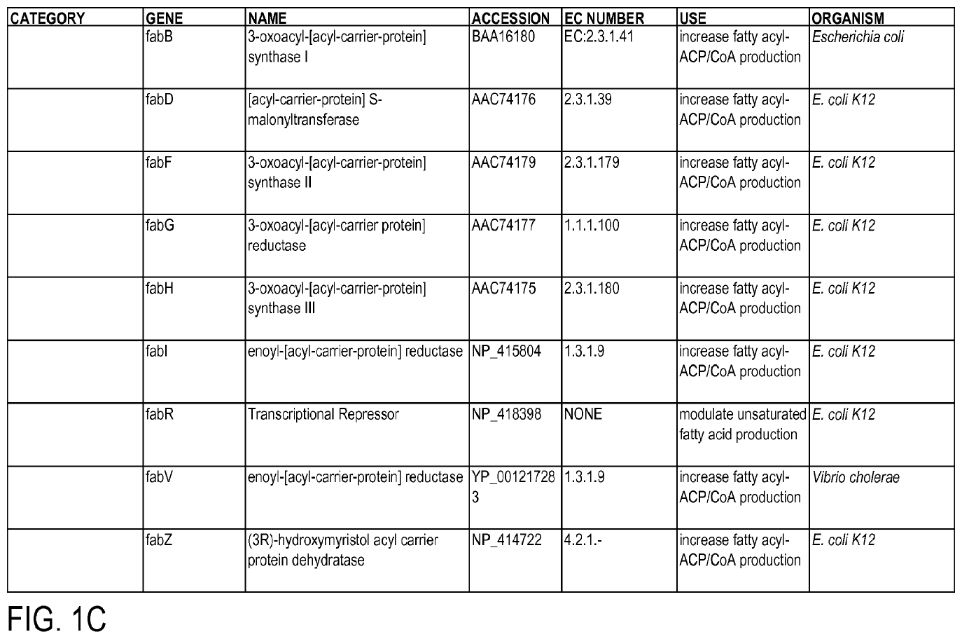

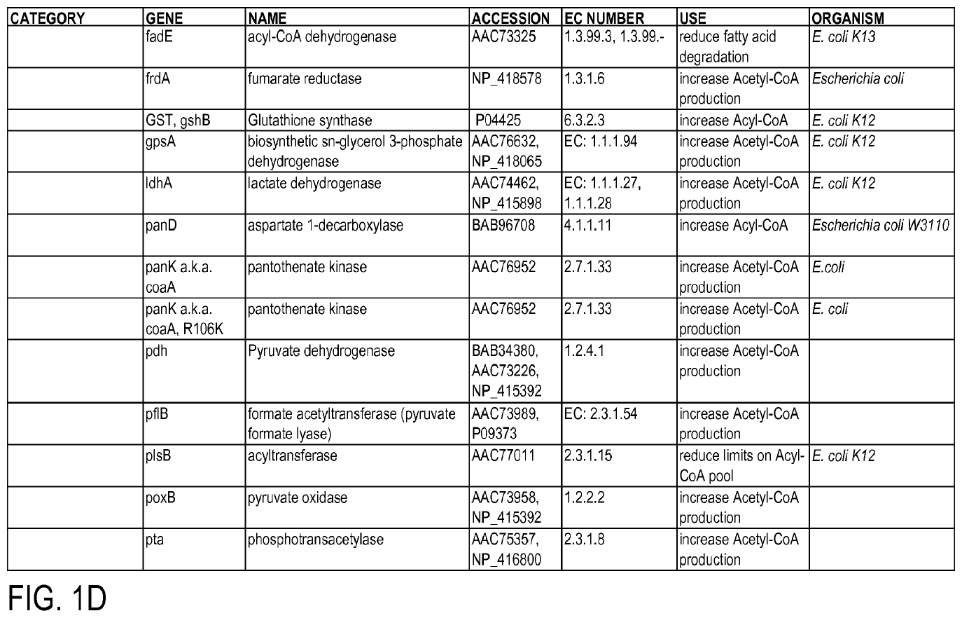

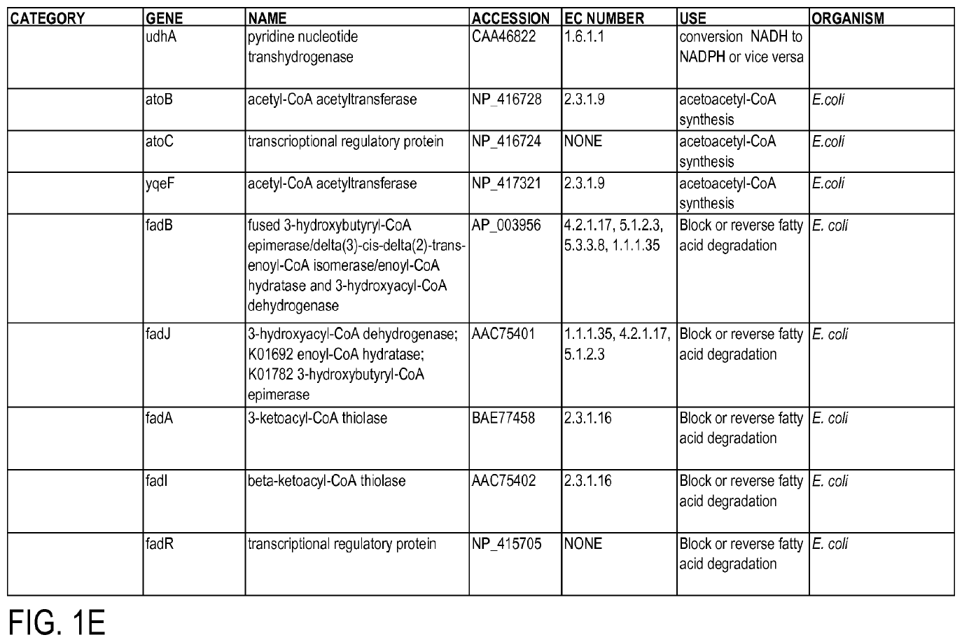

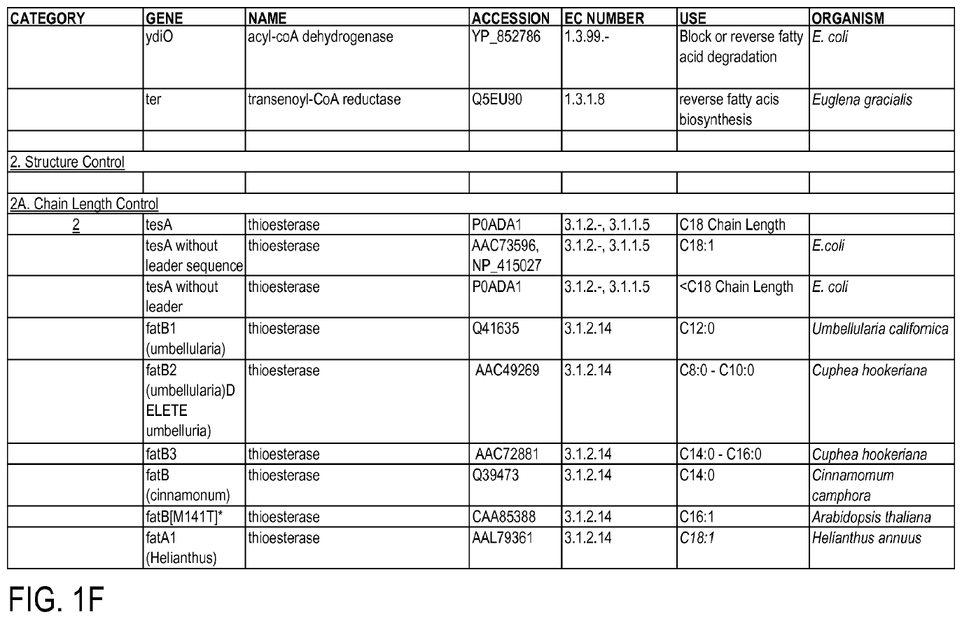

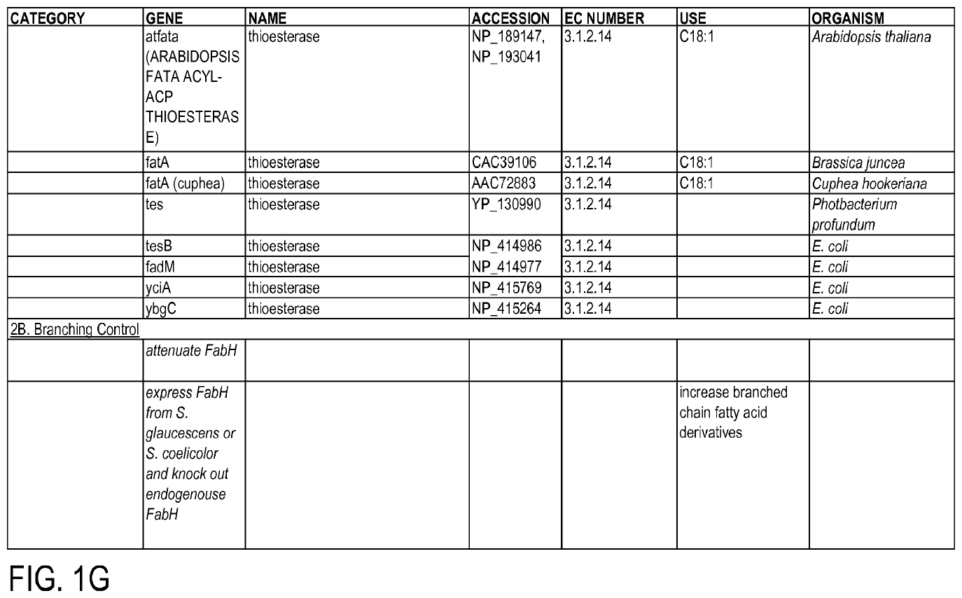

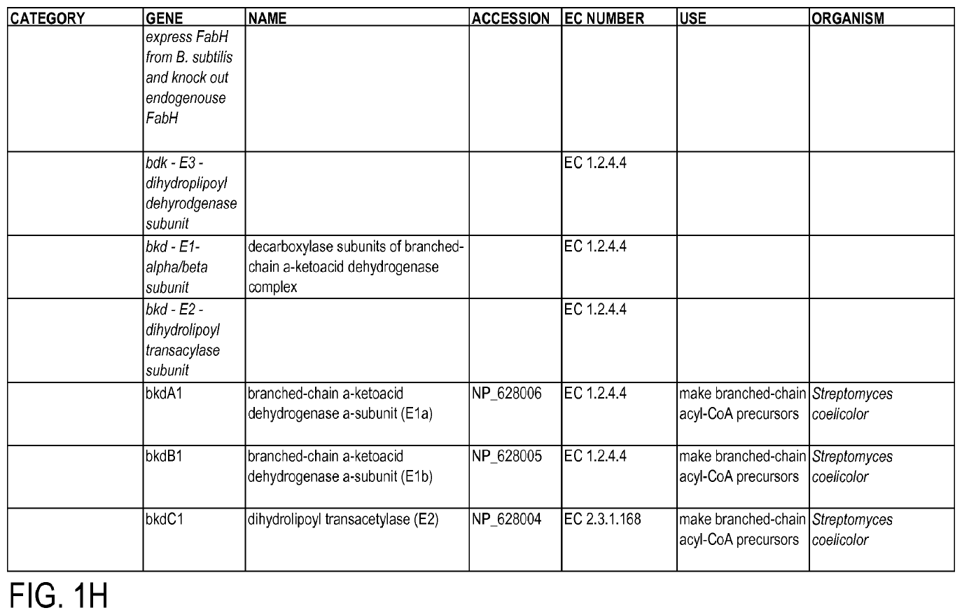

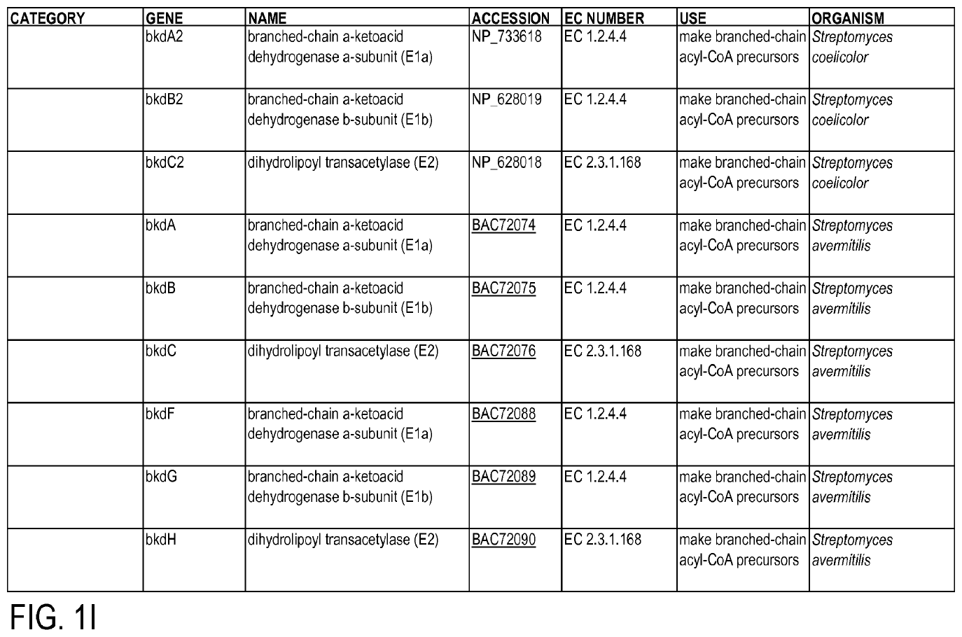

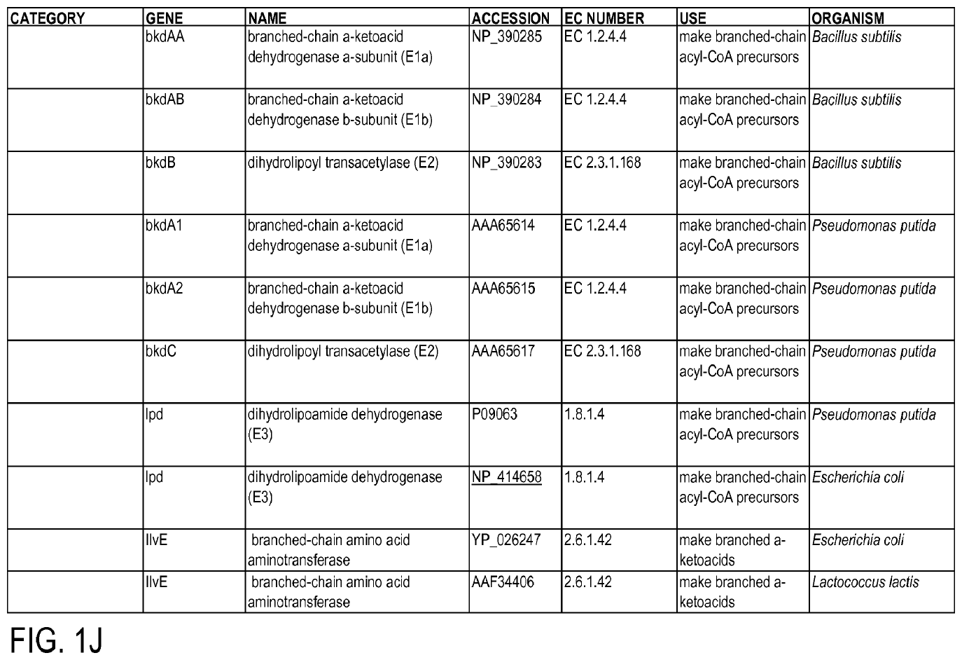

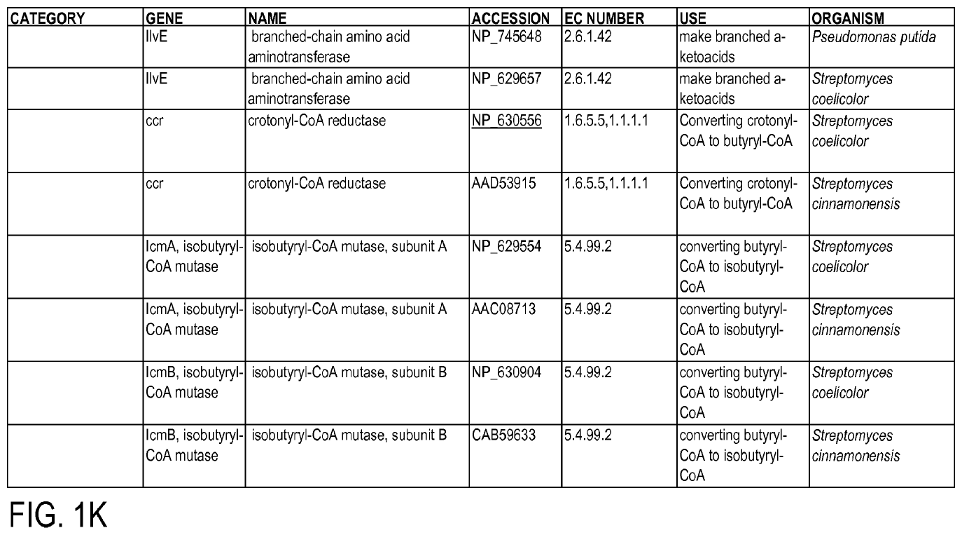

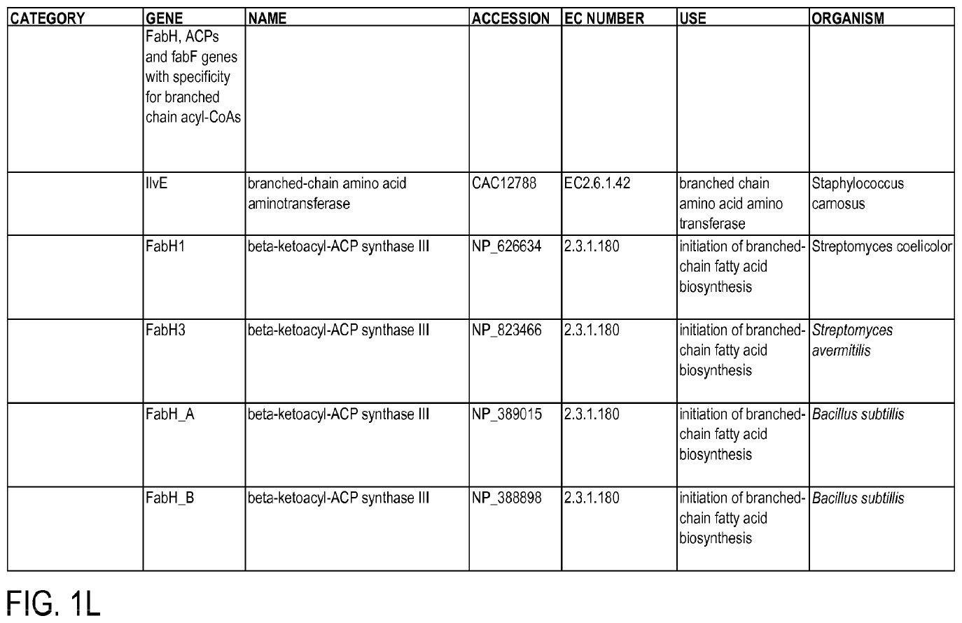

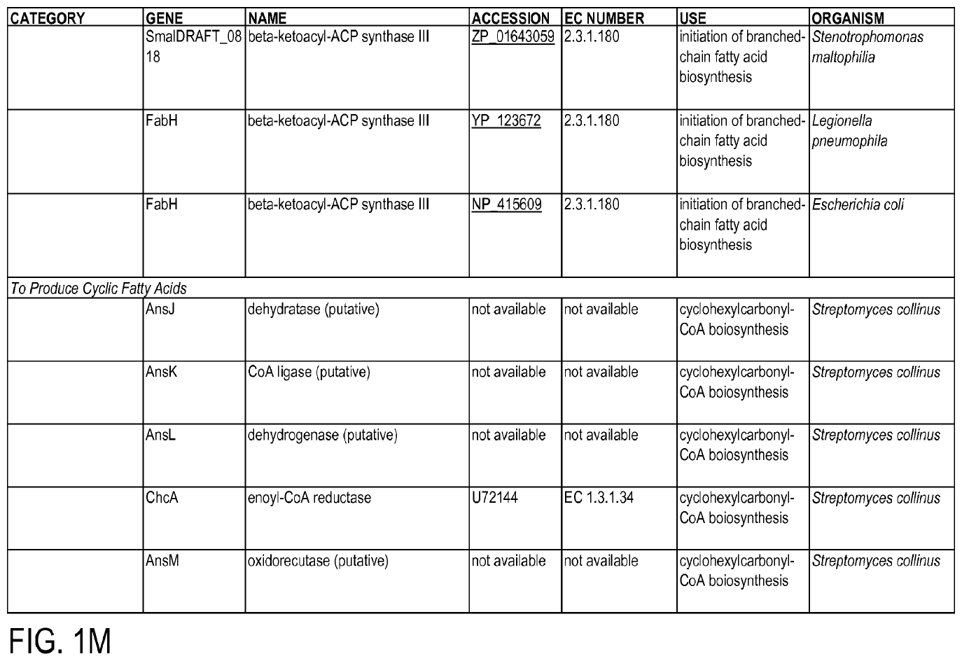

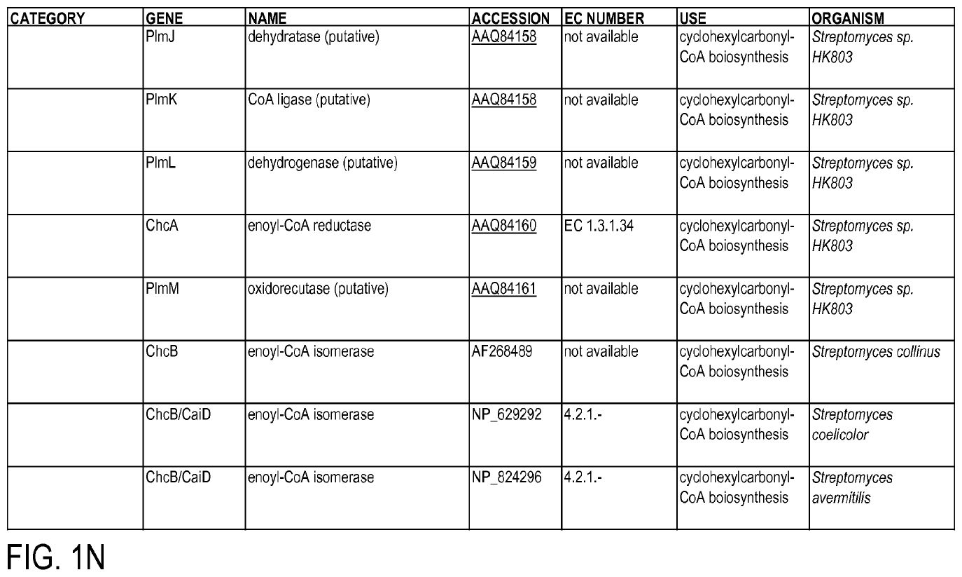

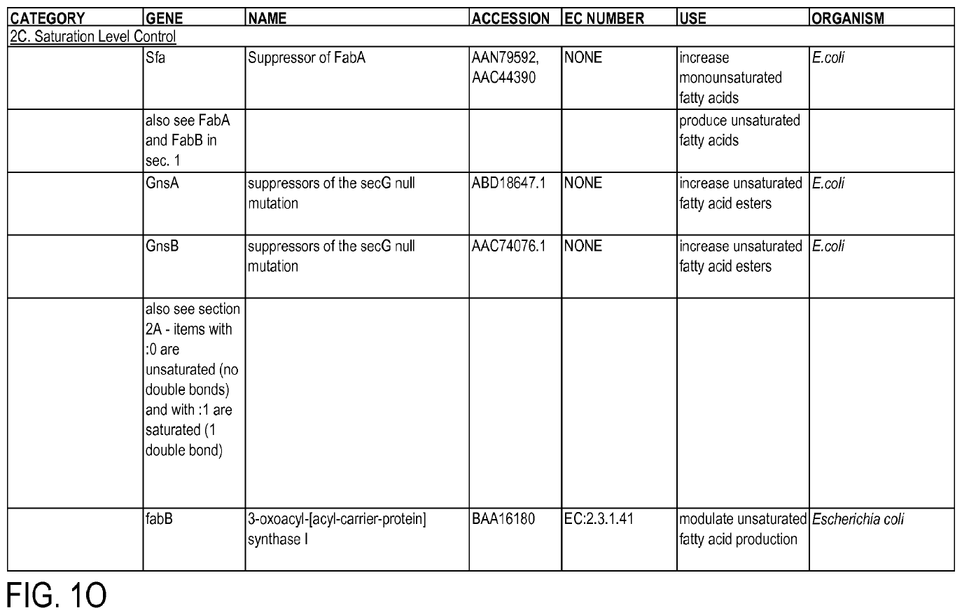

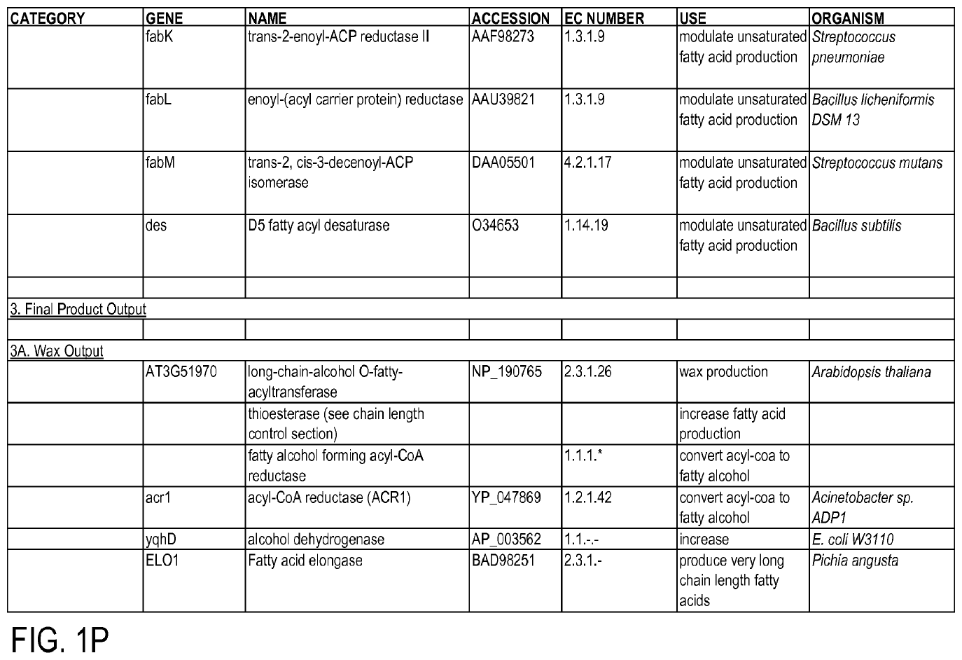

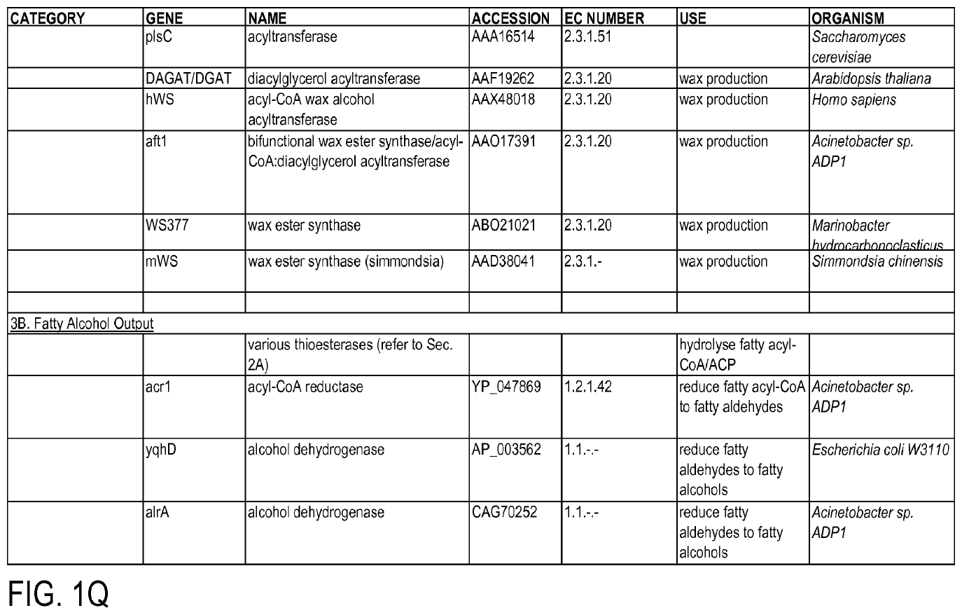

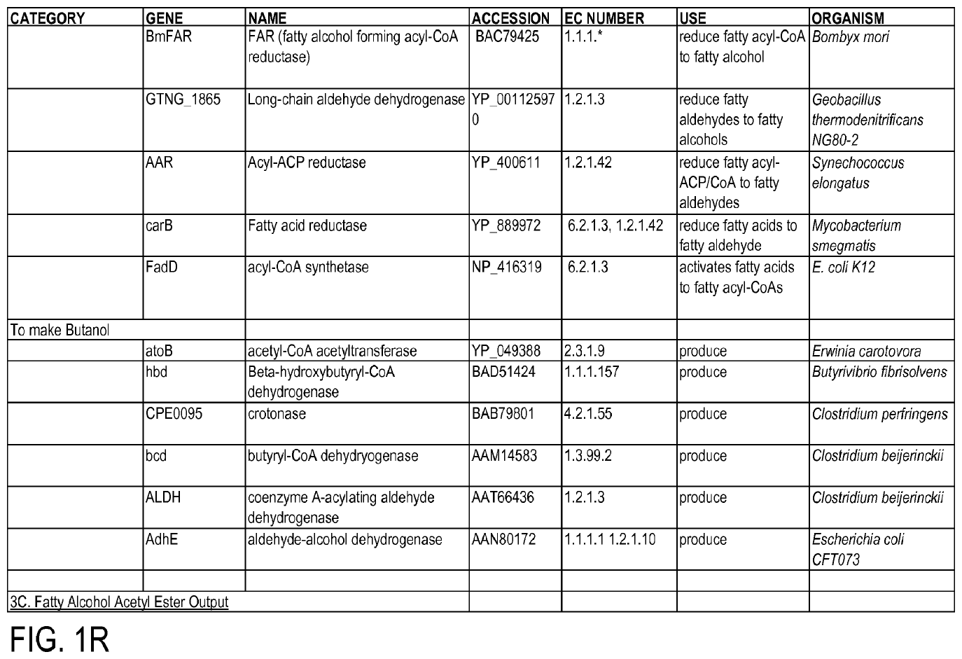

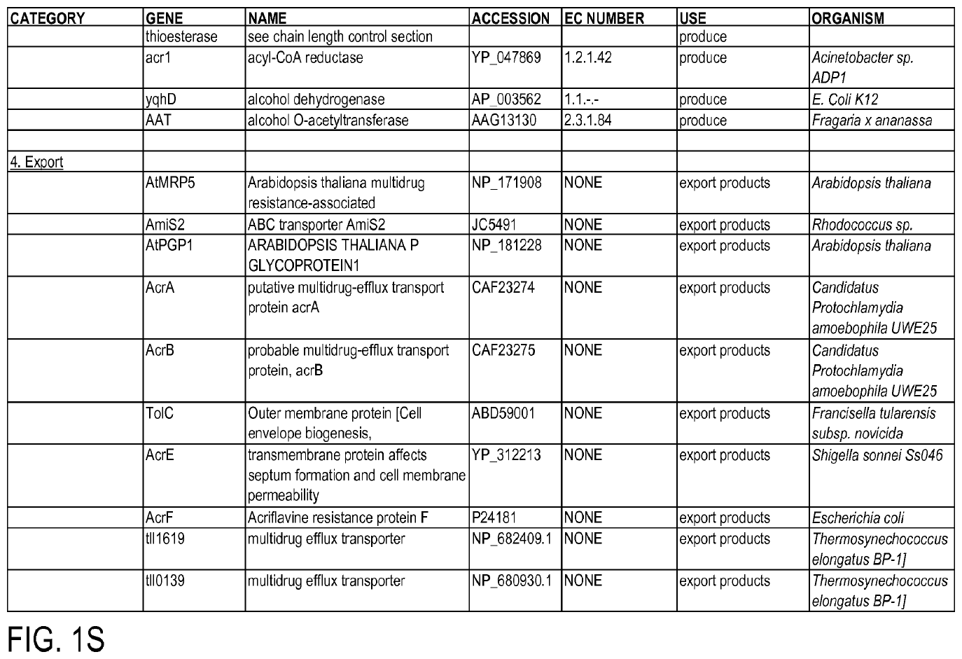

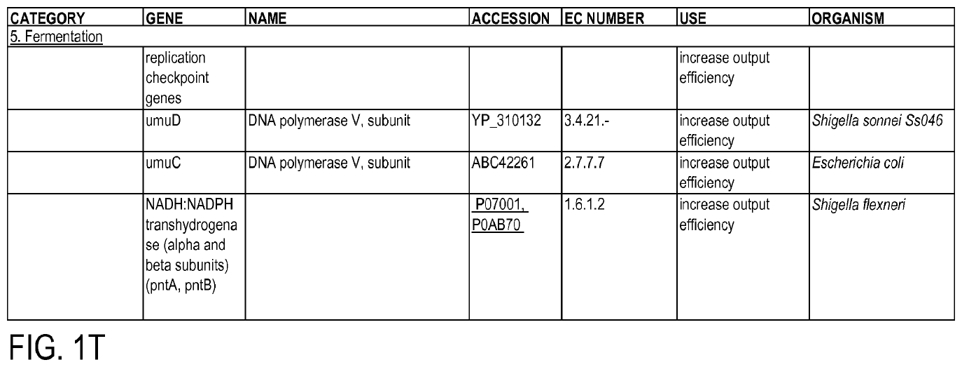

Exemplary enzymes and polypeptides for use in practicing the invention are listed in FIG. 1. One of ordinary skill in the art will understand that depending upon the purpose (e.g., desired fatty acid or fatty acid derivative product), specific genes (or combinations of genes) listed in FIG. 1 may be overexpressed, modified, attenuated or deleted in an engineered host cell which has an altered level of expression of a FadR polypeptide.

In some embodiments, the method comprises modifying the expression of a gene encoding one or more of a thioesterase (e.g., TesA), a decarboxylase, a carboxylic acid reductase (CAR; e.g., CarB), an alcohol dehydrogenase (aldehyde reductase); an aldehyde decarbonylase, a fatty alcohol forming acyl-CoA reductase (FAR), an acyl ACP reductase (AAR), an ester synthase, an acyl-CoA reductase (ACR1), OleA, OleCD and OleBCD.

In certain embodiments of the invention, the engineered host cell having an altered level of expression of a FadR polypeptide may be engineered to further comprise a polynucleotide sequence encoding a polypeptide: (1) having thioesterase activity (EC 3.1.2.14), wherein the engineered host cell synthesizes fatty acids; (2) having decarboxylase activity, wherein the engineered host cell synthesizes terminal olefins; (3) having carboxylic acid reductase activity, wherein the engineered host cell synthesizes fatty aldehydes; (4) having carboxylic acid reductase and alcohol dehydrogenase activity (EC 1.1.1.1), wherein the engineered host cell synthesizes fatty alcohols; (5) having carboxylic acid reductase and aldehyde decarbonylase activity (EC 4.1.99.5), wherein the engineered host cell synthesizes alkanes; (6) having acyl-CoA reductase activity (EC 1.2.1.50), wherein microorganism synthesizes fatty aldehydes; (7) having acyl-CoA reductase activity (EC 1.2.1.50) and alcohol dehydrogenase activity (EC 1.1.1.1), wherein the engineered host cell synthesizes fatty alcohols; (8) having acyl-CoA reductase activity (EC 1.2.1.50) and aldehyde decarbonylase activity (EC 4.1.99.5), wherein the engineered host cell synthesizes alkanes; (9) having alcohol forming acyl CoA reductase activity wherein the engineered host cell synthesizes fatty aldehydes and fatty alcohols; (10) having carboxylic acid reductase activity, wherein the engineered host cell synthesizes fatty aldehydes; (11) having acyl ACP reductase activity, wherein the engineered host cell synthesizes fatty aldehydes; (12) having acyl ACP reductase activity and alcohol dehydrogenase activity (EC 1.1.1.1), wherein engineered host cell synthesizes fatty alcohols; (13) having acyl ACP reductase activity and aldehyde decarbonylase activity (EC 4.1.99.5), wherein engineered host cell synthesizes alkanes; (14) having ester synthase activity (EC 3.1.1.67), wherein the engineered host cell synthesizes fatty esters; (15) having ester synthase activity (EC 3.1.1.67) and (a) carboxylic acid reductase activity, (b) acyl-CoA reductase activity, (c) acyl ACP reductase activity, or (d) alcohol dehydrogenase activity (EC 1.1.1.1), wherein the engineered host cell synthesizes wax esters; (16) having OleA activity, wherein the engineered host cell synthesizes 2-alkyl-3-keto-acyl CoA and ketones; or (17) having OleCD or OleBCD activity, wherein the engineered host cell synthesizes internal olefins.

In some embodiments, the method further comprises modifying the expression of a gene encoding a fatty acid synthase in the host cell. As used herein, “fatty acid synthase” means any enzyme involved in fatty acid biosynthesis. In certain embodiments, modifying the expression of a gene encoding a fatty acid synthase includes expressing a gene encoding a fatty acid synthase in the host cell and/or increasing the expression or activity of an endogenous fatty acid synthase in the host cell. In alternate embodiments, modifying the expression of a gene encoding a fatty acid synthase includes attenuating a gene encoding a fatty acid synthase in the host cell and/or decreasing the expression or activity of an endogenous fatty acid synthase in the host cell. In some embodiments, the fatty acid synthase is a thioesterase (EC 3.1.1.5 or EC 3.1.2.14). In particular embodiments, the thioesterase is encoded by tesA, tesA without leader sequence, tesB, fatB, fatB2, fatB3, fatA, or fatA1.

In other embodiments, the host cell is genetically engineered to express an attenuated level of a fatty acid degradation enzyme relative to a wild-type host cell. As used herein, the term “fatty acid degradation enzyme” means an enzyme involved in the breakdown or conversion of a fatty acid or fatty acid derivative into another product, such as, but not limited to, an acyl-CoA synthase. In some embodiments, the host cell is genetically engineered to express an attenuated level of an acyl-CoA synthase relative to a wild-type host cell. In particular embodiments, the host cell expresses an attenuated level of an acyl-CoA synthase encoded by fadD, fadK, BH3103, yhfl, PJI-4354, EAV15023, fadD1, fadD2, RPC_4074, fadDD35, fadDD22, faa3p, or the gene encoding the protein YP_002028218. In certain embodiments, the genetically engineered host cell comprises a knockout of one or more genes encoding a fatty acid degradation enzyme, such as the aforementioned acyl-CoA synthase genes.

The fatty acid biosynthetic pathway in host cells uses the precursors acetyl-CoA and malonyl-CoA. The steps in this pathway are catalyzed by enzymes of the fatty acid biosynthesis (fab) and acetyl-CoA carboxylase (acc) gene families (see, e.g., Heath et al., Prog. Lipid Res. 40(6): 467-97 (2001)). Acetyl-CoA is carboxylated by acetyl-CoA carboxylase (EC 6.4.1.2), which is a multisubunit enzyme encoded by four separate genes (accA, accB, accC, and accD) in most prokaryotes, to form malonyl-CoA. In some bacteria, such as Corynebacterium glutamicus, acetyl-CoA carboxylase is consisted two subunits, AccDA [YP_225123.1] and AccBC [YP_224991], encoded by accDA and accBC, respectively. Depending upon the desired fatty acid or fatty acid derivative product, specific fab and/or acc genes (or combinations thereof) may be overexpressed, modified, attenuated or deleted in an engineered host cell.

In some embodiments, an acetyl-CoA carboxylase complex is overexpressed in the engineered host cell. In certain embodiments, the acetyl-CoA carboxylase subunit genes are obtained from one or more of Corynebacterium glutamicum, Escherichia coli, Lactococcus lactis, Kineococcus radiotolerans, Desulfovibrio desulfuricans, Erwinia amylovora, Rhodospirillum rubrum, Vibrio furnissii, Stenotrophomonas maltophilia, Synechocystis sp. PCC6803, and Synechococcus elongatus.

Biotin protein ligase (EC 6.3.4.15) is an enzyme that catalyzes the covalent attachment of biotin to the biotin carboxyl carrier protein (BCCP) subunit of acetyl-CoA carboxylase. In some embodiments of the present invention, a biotin protein ligase is expressed or overexpressed in the engineered host cell. In certain embodiments, the biotin protein ligase is birA from Corynebacterium glutamicum (YP_225000) or bpl1 from Saccharomyces cerevisiae (NP_010140).

The production of fatty acid esters such as FAMEs or FAEEs in a host cell can be facilitated by expression or overexpression of an ester synthase (EC 2.3.1.75 or EC 3.1.1.67) in an engineered host cell. In some embodiments, the ester synthase is ES9 from Marinobacter hydrocarbonoclasticus (SEQ ID NO: 2), ES8 from Marinobacter hydrocarbonoclasticus (SEQ ID NO: 3), AtfA1 from Alcanivorax borkumensis SK2 (SEQ ID NO: 4), AtfA2 from Alcanivorax borkumensis SK2 (SEQ ID NO: 5), diacylglycerol O-acyltransferase from Marinobacter aquaeolei VT8 (SEQ ID NO: 6 or SEQ ID NO: 7), a wax synthase, or a bifunctional wax ester synthase/acyl-CoA: diacylglycerol acyltransferase (wax-dgaT).

In certain embodiments, a gene encoding a fatty aldehyde biosynthetic polypeptide is expressed or overexpressed in the host cell. Exemplary fatty aldehyde biosynthetic polypeptides suitable for use in the methods of the invention are disclosed, for example, in International Patent Application Publication WO 2010/042664. In preferred embodiments, the fatty aldehyde biosynthetic polypeptide has carboxylic acid reductase (EC 6.2.1.3 or EC 1.2.1.42) activity, e.g., fatty acid reductase activity.

In the methods of the invention, the polypeptide having carboxylic acid reductase activity is not particularly limited. Exemplary polypeptides having carboxylic acid reductase activity which are suitable for use in the methods of the present invention are disclosed, for example, in International Patent Application Publications WO 2010/062480 and WO 2010/042664. In some embodiments, the polypeptide having carboxylic acid reductase activity is CarB from M. smegmatis (YP_889972) (SEQ ID NO: 8). In other embodiments, the polypeptide having carboxylic acid reductase activity is CarA [ABK75684] from M. smegmatis, FadD9 [AAK46980] from M. tuberculosis, CAR [AAR91681] from Nocardia sp. NRRL 5646, CAR [YP-001070587] from Mycobacterium sp. JLS, or CAR [YP-118225] from Streptomyces griseus. The terms “carboxylic acid reductase,” “CAR,” and “fatty aldehyde biosynthetic polypeptide” are used interchangeably herein.

In certain embodiments, a thioesterase and a carboxylic acid reductase are expressed or overexpressed in the engineered host cell.

In some embodiments, a gene encoding a fatty alcohol biosynthetic polypeptide is expressed or overexpressed in the host cell. Exemplary fatty alcohol biosynthetic polypeptides suitable for use in the methods of the invention are disclosed, for example, in International Patent Application Publication WO 2010/062480. In certain embodiments, the fatty alcohol biosynthetic polypeptide has aldehyde reductase or alcohol dehydrogenase activity (EC 1.1.1.1). Exemplary fatty alcohol biosynthetic polypeptides include, but are not limited to AlrA of Acenitobacter sp. M-1 (SEQ ID NO: 9) or AlrA homologs and endogenous E. coli alcohol dehydrogenases such as YjgB, (AAC77226) (SEQ ID NO: 10), DkgA (NP_417485), DkgB (NP_414743), YdjL (AAC74846), YdjJ (NP_416288), AdhP (NP_415995), YhdH (NP_417719), YahK (NP_414859), YphC (AAC75598), YqhD (446856) and YbbO [AAC73595.1].

As used herein, the term “alcohol dehydrogenase” is a peptide capable of catalyzing the conversion of a fatty aldehyde to an alcohol (e.g., fatty alcohol). One of ordinary skill in the art will appreciate that certain alcohol dehydrogenases are capable of catalyzing other reactions as well. For example, certain alcohol dehydrogenases will accept other substrates in addition to fatty aldehydes, and these non-specific alcohol dehydrogenases also are encompassed by the term “alcohol dehydrogenase.” Exemplary alcohol dehydrogenases suitable for use in the methods of the invention are disclosed, for example, in International Patent Application Publication WO 2010/062480.

In some embodiments, a thioesterase, a carboxylic acid reductase, and an alcohol dehydrogenase are expressed or overexpressed in the engineered host cell. In certain embodiments, the thioesterase is tesA (SEQ ID NO: 11), the carboxylic acid reductase is carB (SEQ ID NO: 8), and the alcohol dehydrogenase is YjgB (SEQ ID NO: 10) or AlrAadp1 (SEQ ID NO: 9).

Phosphopantetheine transferases (PPTases) (EC 2.7.8.7) catalyze the transfer of 4′-phosphopantetheine from CoA to a substrate. Nocardia CAR and several of its homologues contain a putative attachment site for 4′-phosphopantetheine (PPT) (He et al., Appl. Environ. Microbiol., 70(3): 1874-1881 (2004)). In some embodiments of the invention, a PPTase is expressed or overexpressed in an engineered host cell. In certain embodiments, the PPTase is EntD from E. coli MG1655 (SEQ ID NO: 12).

In some embodiments, a thioesterase, a carboxylic acid reductase, a PPTase, and an alcohol dehydrogenase are expressed or overexpressed in the engineered host cell. In certain embodiments, the thioesterase is tesA (SEQ ID NO: 11), the carboxylic acid reductase is carB (SEQ ID NO: 8), the PPTase is entD (SEQ ID NO: 12), and the alcohol dehydrogenase is yjgB (SEQ ID NO: 10) or alrAadp1 (SEQ ID NO: 9).

The invention also provides a fatty acid or a fatty derivative produced by any of the methods described herein. A fatty acid or derivative thereof produced by any of the methods described herein can be used directly as fuels, fuel additives, starting materials for production of other chemical compounds (e.g., polymers, surfactants, plastics, textiles, solvents, adhesives, etc.), or personal care additives. These compounds can also be used as feedstock for subsequent reactions, for example, hydrogenation, catalytic cracking (e.g., via hydrogenation, pyrolisis, or both), to make other products.

In some embodiments, the invention provides a biofuel composition comprising the fatty acid or derivative thereof produced by the methods described herein. As used herein, the term “biofuel” refers to any fuel derived from biomass. Biofuels can be substituted for petroleum-based fuels. For example, biofuels are inclusive of transportation fuels (e.g., gasoline, diesel, jet fuel, etc.), heating fuels, and electricity-generating fuels. Biofuels are a renewable energy source. As used herein, the term “biodiesel” means a biofuel that can be a substitute of diesel, which is derived from petroleum. Biodiesel can be used in internal combustion diesel engines in either a pure form, which is referred to as “neat” biodiesel, or as a mixture in any concentration with petroleum-based diesel. Biodiesel can include esters or hydrocarbons, such as alcohols. In certain embodiments, the biofuel is selected from the group consisting of a biodiesel, a fatty alcohol, a fatty ester, a triacylglyceride, a gasoline, or a jet fuel.

Fuel additives are used to enhance the performance of a fuel or engine. For example, fuel additives can be used to alter the freezing/gelling point, cloud point, lubricity, viscosity, oxidative stability, ignition quality, octane level, and/or flash point of a fuel. In the United States, all fuel additives must be registered with Environmental Protection Agency (EPA). The names of fuel additives and the companies that sell the fuel additives are publicly available by contacting the EPA or by viewing the EPA's website. One of ordinary skill in the art will appreciate that a biofuel produced according to the methods described herein can be mixed with one or more fuel additives to impart a desired quality.

The invention also provides a surfactant composition or a detergent composition comprising a fatty alcohol produced by any of the methods described herein. One of ordinary skill in the art will appreciate that, depending upon the intended purpose of the surfactant or detergent composition, different fatty alcohols can be produced and used. For example, when the fatty alcohols described herein are used as a feedstock for surfactant or detergent production, one of ordinary skill in the art will appreciate that the characteristics of the fatty alcohol feedstock will affect the characteristics of the surfactant or detergent composition produced. Hence, the characteristics of the surfactant or detergent composition can be selected for by producing particular fatty alcohols for use as a feedstock.

A fatty alcohol-based surfactant and/or detergent composition described herein can be mixed with other surfactants and/or detergents well known in the art. In some embodiments, the mixture can include at least about 10%, at least about 15%, at least about 20%, at least about 30%, at least about 40%, at least about 50%, at least about 60%, or a range bounded by any two of the foregoing values, by weight of the fatty alcohol. In other examples, a surfactant or detergent composition can be made that includes at least about 5%, at least about 10%, at least about 20%, at least about 30%, at least about 40%, at least about 50%, at least about 60%, at least about 70%, at least about 80%, at least about 85%, at least about 90%, at least about 95%, or a range bounded by any two of the foregoing values, by weight of a fatty alcohol that includes a carbon chain that is 8, 9, 10, 11, 12, 13, 14, 15, 16, 17, 18, 19, 20, 21, or 22 carbons in length. Such surfactant or detergent compositions also can include at least one additive, such as a microemulsion or a surfactant or detergent from nonmicrobial sources such as plant oils or petroleum, which can be present in the amount of at least about 5%, at least about 10%, at least about 15%, at least about 20%, at least about 30%, at least about 40%, at least about 50%, at least about 60%, at least about 70%, at least about 80%, at least about 85%, at least about 90%, at least about 95%, or a range bounded by any two of the foregoing values, by weight of the fatty alcohol.

Bioproducts (e.g., fatty acids, acyl-CoAs, hydrocarbons, fatty aldehydes, fatty alcohols, fatty esters, surfactant compositions, and biofuel compositions) produced according to the methods of the invention can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting (see, e.g., U.S. Pat. No. 7,169,588).

The ability to distinguish bioproducts from petroleum-based organic compounds is beneficial in tracking these materials in commerce. For example, organic compounds or chemicals comprising both biologically-based and petroleum-based carbon isotope profiles may be distinguished from organic compounds and chemicals made only of petroleum-based materials. Hence, the materials prepared in accordance with the inventive methods may be followed in commerce on the basis of their unique carbon isotope profile.

Bioproducts can be distinguished from petroleum-based organic compounds by comparing the stable carbon isotope ratio (¹³C/¹²C) in each fuel. The ¹³C/¹²C ratio in a given bioproduct is a consequence of the ¹³C/¹²C ratio in atmospheric carbon dioxide at the time the carbon dioxide is fixed. It also reflects the precise metabolic pathway. Regional variations also occur. Petroleum, C₃ plants (the broadleaf), C₄ plants (the grasses), and marine carbonates all show significant differences in ¹³C/¹²C and the corresponding δ¹³C values. Furthermore, lipid matter of C₃ and C₄ plants analyze differently than materials derived from the carbohydrate components of the same plants as a consequence of the metabolic pathway.

The ¹³C measurement scale was originally defined by a zero set by Pee Dee Belemnite (PDB) limestone, where values are given in parts per thousand deviations from this material. The “δ¹³C” values are expressed in parts per thousand (per mil), abbreviated, % o, and are calculated as follows:

$${{{{δ}^{13} C ( \% o ) = [ {(}^{13} C {/}^{12} C )}_{sample} - {(}^{13} C {/}^{12} C )}_{standard} ] / {(}^{13} C {/}^{12} C )}_{standard} \times 1000$$

In some embodiments, a bioproduct produced according to the methods of the invention has a δ¹³C of about −30 or greater, about −28 or greater, about −27 or greater, about −20 or greater, about −18 or greater, about −15 or greater, about −13 or greater, or about −10 or greater. Alternatively, or in addition, a bioproduct has a δ¹³C of about−4 or less, about −5 or less, about −8 or less, about −10 or less, about −13 or less, about −15 or less, about −18 or less, or about −20 or less. Thus, the bioproduct can have a δ¹³C bounded by any two of the above endpoints. For example, the bioproduct can have a δ¹³C of about −30 to about −15, about −27 to about −19, about −25 to about −21, about −15 to about −5, about −13 to about −7, or about −13 to about −10. In some embodiments, the bioproduct can have a δ¹³C of about −10, −11, −12, or −12.3. In other embodiments, the bioproduct has a δ¹³C of about −15.4 or greater. In yet other embodiments, the bioproduct has a δ¹³C of about −15.4 to about −10.9, or a δ¹³C of about −13.92 to about −13.84.

Bioproducts can also be distinguished from petroleum-based organic compounds by comparing the amount of ¹⁴C in each compound. Because ¹⁴C has a nuclear half life of 5730 years, petroleum based fuels containing “older” carbon can be distinguished from bioproducts which contain “newer” carbon (see, e.g., Currie, “Source Apportionment of Atmospheric Particles”, Characterization of Environmental Particles, J. Buffle and H. P. van Leeuwen, Eds., Vol. I of the IUPAC Environmental Analytical Chemistry Series, Lewis Publishers, Inc., pp. 3-74 (1992)).

¹⁴C can be measured by accelerator mass spectrometry (AMS), with results given in units of “fraction of modern carbon” (f_{M}). f_{M} is defined by National Institute of Standards and Technology (NIST) Standard Reference Materials (SRMs) 4990B and 4990C. As used herein, “fraction of modem carbon” or f_{M} has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

In some embodiments, a bioproduct produced according to the methods of the invention has a f_{M}¹⁴C. of at least about 1, e.g., at least about 1.003, at least about 1.01, at least about 1.04, at least about 1.111, at least about 1.18, or at least about 1.124. Alternatively, or in addition, the bioproduct has an f_{M}¹⁴C of about 1.130 or less, e.g., about 1.124 or less, about 1.18 or less, about 1.111 or less, or about 1.04 or less. Thus, the bioproduct can have a f_{M}¹⁴C bounded by any two of the above endpoints. For example, the bioproduct can have a f_{M}¹⁴C of about 1.003 to about 1.124, a f_{M}¹⁴C of about 1.04 to about 1.18, or a f_{M}¹⁴C of about 1.111 to about 1.124.

Another measurement of ¹⁴C is known as the percent of modem carbon, i.e., pMC. For an archaeologist or geologist using 14C dates, AD 1950 equals “zero years old.” This also represents 100 pMC. “Bomb carbon” in the atmosphere reached almost twice the normal level in 1963 at the peak of thermo-nuclear weapons testing. Its distribution within the atmosphere has been approximated since its appearance, showing values that are greater than 100 pMC for plants and animals living since AD 1950. It has gradually decreased over time with today's value being near 107.5 pMC. This means that a fresh biomass material, such as corn, would give a ¹⁴C signature near 107.5 pMC. Petroleum-based compounds will have a pMC value of zero. Combining fossil carbon with present day carbon will result in a dilution of the present day pMC content. By presuming 107.5 pMC represents the 14C content of present day biomass materials and 0 pMC represents the ¹⁴C content of petroleum-based products, the measured pMC value for that material will reflect the proportions of the two component types. For example, a material derived 100% from present day soybeans would have a radiocarbon signature near 107.5 pMC. If that material was diluted 50% with petroleum-based products, the resulting mixture would have a radiocarbon signature of approximately 54 pMC.

A biologically-based carbon content is derived by assigning “100%” equal to 107.5 pMC and “0%” equal to 0 pMC. For example, a sample measuring 99 pMC will provide an equivalent biologically-based carbon content of 93%. This value is referred to as the mean biologically-based carbon result and assumes that all of the components within the analyzed material originated either from present day biological material or petroleum-based material.

In some embodiments, a bioproduct produced according to the methods of the invention has a pMC of at least about 50, at least about 60, at least about 70, at least about 75, at least about 80, at least about 85, at least about 90, at least about 95, at least about 96, at least about 97, or at least about 98. Alternatively, or in addition, the bioproduct has a pMC of about 100 or less, about 99 or less, about 98 or less, about 96 or less, about 95 or less, about 90 or less, about 85 or less, or about 80 or less. Thus, the bioproduct can have a pMC bounded by any two of the above endpoints. For example, a bioproduct can have a pMC of about 50 to about 100; about 60 to about 100; about 70 to about 100; about 80 to about 100; about 85 to about 100; about 87 to about 98; or about 90 to about 95. In other embodiments, a bioproduct described herein has a pMC of about 90, about 91, about 92, about 93, about 94, or about 94.2.

The following examples further illustrate the invention but, of course, should not be construed as in any way limiting its scope.

### EXAMPLE 1

This example demonstrates a method to identify engineered host cells which display enhanced production of fatty acids and derivatives thereof.

ALC310 is a previously characterized E. coli strain having the genotype MG1655 (ΔfadE::FRT ΔfhuA::FRT fabB[A329V] ΔentD::P_{T}5-entD) which carries the plasmid ALC310 (pCL1920_P_{TRC}_carBopt_13G04_alrA_sthA) (SEQ ID NO: 13) and produces fatty acids and derivatives thereof. To identify strains which display an improved titer or yield of fatty acids or derivatives thereof, transposon mutagenesis of ALC310 was performed followed by high-throughput screening.

The transposon DNA was prepared by cloning a DNA fragment into the plasmid EZ-Tn5™ pMOD™<R6K ori/MCS> (Epicentre Biotechnologies, Madison, WI). The DNA fragment contains a T5 promoter and a chloramphenicol resistance gene (cat) flanked by loxP sites. The resulting plasmid was named p100.38 (SEQ ID NO: 14). The p100.38 plasmid was optionally digested with PshAI restriction enzyme, incubated with EZ-Tn5™ Transposase enzyme (Epicentre Biotechnologies, Madison, WI), and electroporated into electrocompetent ALC310 cells as per the manufacturer's instructions. The resulting colonies contained the transposon DNA inserted randomly into the chromosome of ALC310.

Transposon clones were then subjected to high-throughput screening to measure production of fatty alcohols. Briefly, colonies were picked into deep-well plates containing Luria-Bertani (LB) medium. After overnight growth, each culture was inoculated into fresh LB. After 3 hours growth, each culture was inoculated into fresh FA-2 media. Spectinomycin (100 μg/mL) was included in all media to maintain selection of the 7P36 plasmid. FA-2 medium is M9 medium with 3% glucose supplemented with antibiotics, 10 μg/L iron citrate, 1 μg/L thiamine, 0.1 M Bis-Tris buffer (pH 7.0), and a 1:1000 dilution of the trace mineral solution described in Table 1.

TABLE 1
Trace mineral solution
(filter sterilized)
2 g/L ZnCl•4H₂O
2 g/L CaCl₂•6H₂O
2 g/L Na₂MoO₄•2H₂O
1.9 g/L CuSO₄•5H₂O
0.5 g/L H₃BO₃
100 mL/L concentrated HCl
q.s. Milli-Q water

After 20 hours growth in FA-2, the cultures were extracted with butyl acetate. The crude extract was derivatized with BSTFA (N,O-bis [Trimethylsilyl]trifluoroacetamide) and total fatty species (e.g., fatty alcohols, fatty aldehydes, and fatty acids) were measured with GC-FID as described in International Patent Application Publication WO 2008/119082.

Clones that produced 15% more total fatty species than ALC310 were subjected to further verification using a shake-flask fermentation. Briefly, the clones were grown in 2 mL of LB medium supplemented with spectinomycin (100 mg/L) at 37° C. After overnight growth, 100 μL of culture was transferred into 2 mL of fresh LB supplemented with antibiotics. After 3 hours growth, 2 mL of culture was transferred into a 125 mL-flask containing 18 mL of FA-2 medium supplemented with spectinomycin (100 mg/L). When the OD₆₀₀ of the culture reached 2.5, 1 mM of IPTG was added to each flask. After 20 hours of growth at 37° C., a 400 μL sample from each flask was removed, and total fatty species were extracted with 400 μL butyl acetate. The crude extracts were analyzed directly with GC-FID as described above.

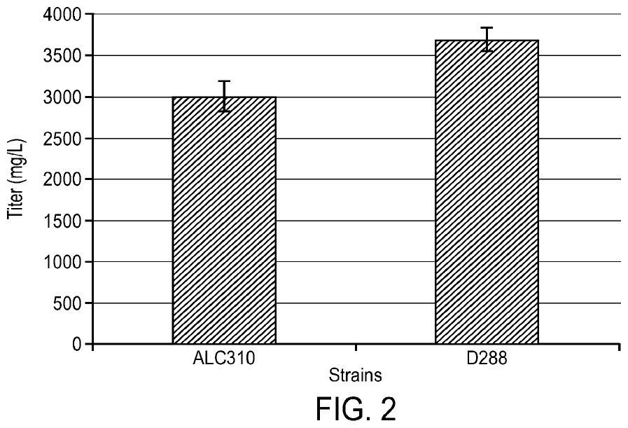

A transposon clone (termed D288) was identified which displayed increased titers of total fatty species as compared to the parental ALC310 strain (FIG. 2).

The results of this example demonstrate a method to identify engineered host cells which display enhanced production of fatty acids and derivatives thereof as compared to a corresponding wild-type host cell.

### EXAMPLE 2

This example demonstrates that an engineered host cell with a transposon insertion in the nhaB gene displays enhanced production of fatty acids and derivatives thereof as compared to a corresponding wild-type host cell.

Sequence analysis was performed to identify the location of the transposon insertion in the D288 strain identified in Example 1. To do so, genomic DNA was purified from a 3-mL overnight LB culture of D288 cells using the ZR Fungal/Bacterial DNA MiniPrep™ kit (Zymo Research) according to the manufacturer's instructions. The purified genomic DNA was sequenced outward from the transposon using the primers DG150 (GCAGTTATTGGTGCCCTTAAACGCCTGGTTGCTACGCCTG) (SEQ ID NO: 15) and DG153 (CCCAGGGCTTCCCGGTATCAACAGGGACACCAGG) (SEQ ID NO: 16), internal to the transposon.

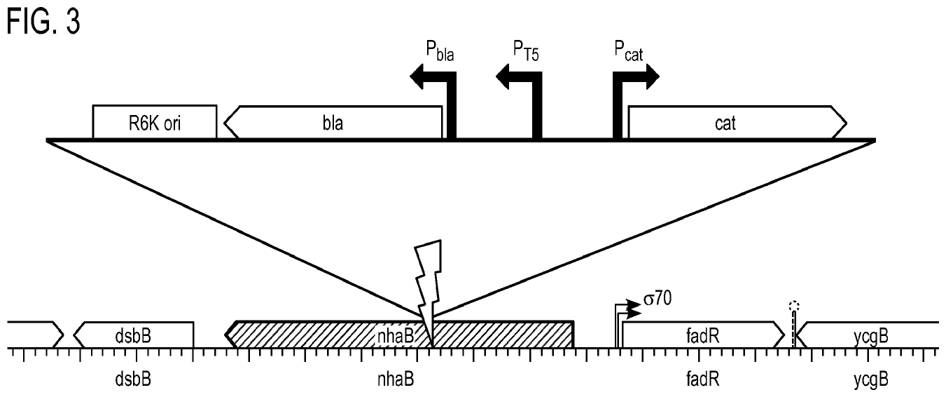

The D288 strain was determined to have a transposon insertion in the nhaB gene (FIG. 3).

The results of this example demonstrate an engineered E. coli host cell with a transposon insertion in the nhaB gene displays enhanced production of fatty acids and derivatives thereof as compared to a corresponding wild-type E. coli host cell.

### EXAMPLE 3

This example demonstrates that engineered host cells having an altered level of production of FadR display enhanced production of fatty acids and derivatives thereof.

The nhaB gene is proximal to the gene encoding the fatty acid degradation regulator, FadR (FIG. 3). To determine if altering the expression of FadR affects the production of fatty acids or derivatives thereof in host cells, a FadR expression library was cloned and screened.

To clone the expression library, the wild-type fadR gene was amplified from genomic DNA of E. coli MG1655 by PCR using primers DG191 (SEQ ID NO: 17) and DG192 (SEQ ID NO: 18). A mutant fadR gene containing amino acid change S219N was also amplified from E. coli MG1655 fadR[S219N] genomic DNA using the DG191 and DG192 primer set. The primers used in this example are listed in Table 2.

TABLE 2

| | | Sequence |
|---|---|---|
| Primer | Sequence | Identifier |
| DG191 | ATGGTCATTAAGGCGCAAAGCCCGG | SEQ ID NO: 17 |
| DG192 | GAGACCCCACACTACCATCCTCGAGTTATCGCCCCTGA | SEQ ID NO: 18 |
| | ATGGCTAAATCACCC | |
| SL03 | CTCGAGGATGGTAGTGTGGGGTCTCCC | SEQ ID NO: 19 |
| SL23 | GAGACCGTTTCTCGAATTTAAATATGATACGCTCGAGCT | SEQ ID NO: 20 |
| | TCGTCTGTTTCTACTGGTATTGGCACAAAC | |
| DG193 | TGAAAGATTAAATTTNHHARNDDHDDNWAGGAGNNNN | SEQ ID NO: 21 |
| | NNNATGGTCATTAAGGCGCAAAGCCCGG | |

A gene cassette encoding for kanamycin resistance (kan) was PCR amplified from plasmid pKD13 using primers SL03 (SEQ ID NO: 19) and SL23 (SEQ ID NO: 20). Each fadR cassette (i.e., wild-type and S219N mutant) was separately joined with the kanamycin resistance cassette using splicing by overlap extension (SOE) PCR using primers SL23 and DG193 (SEQ ID NO: 21). The DG193 primer contained degenerate nucleotides for the generation of expression variants.

Plasmid p100.487 (pCL1920_PTRC_carBopt_13G04_alrA_fabB [A329G]) (SEQ ID NO: 22) was linearized via restriction digestion with enzymes Swal and Xhol. Each of the two SOE PCR fadR-kan products were separately cloned into linearized plasmid p100.487 using the INFUSION™ system (Clontech, Mountain View, CA), and then the plasmids were transformed into chemically competent NEB TURBO™ cells (New England Biolabs, Ipswich, MA). Transformants were plated on LB agar containing 50 μg/mL kanamycin.

Thousands of colonies were obtained for fadR and fadR [S219N]. Colonies were scraped from plates and the plasmids were isolated by miniprepping according to standard protocols. The resulting pool of plasmids was transformed into an E. coli EG149 strain having a genotype of MG1655 (ΔfadE::FRT ΔfhuA::FRT fabB[A329V] ΔentD::P_{T5}-entD)), and selected on LB plates containing 100 μg/mL spectinomycin.

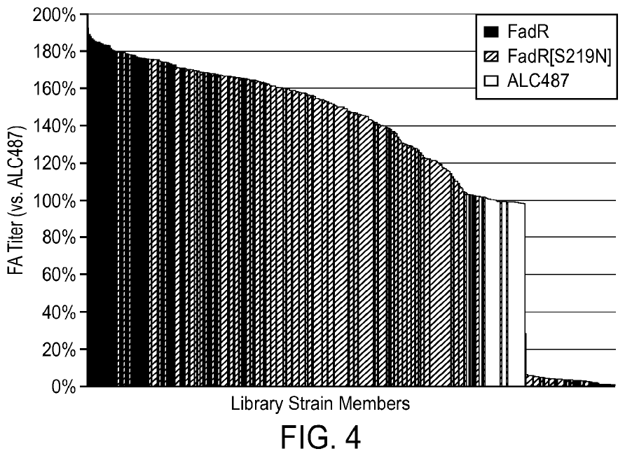

Transformants were then screened for production of total fatty species (e.g., fatty acids, fatty aldehydes, and fatty alcohols) using the deep-well procedure described in Example 1. Numerous strains were identified which displayed enhanced production of total fatty species as compared to the control ALC487 strain (EG149 strain carrying plasmid p100.487) (FIG. 4). Strains expressing either wild-type fadR or fadR [S219N] displayed enhanced production of total fatty species as compared to the ALC487 strain, although the highest titers were observed in strains expressing wild-type FadR (FIG. 4).

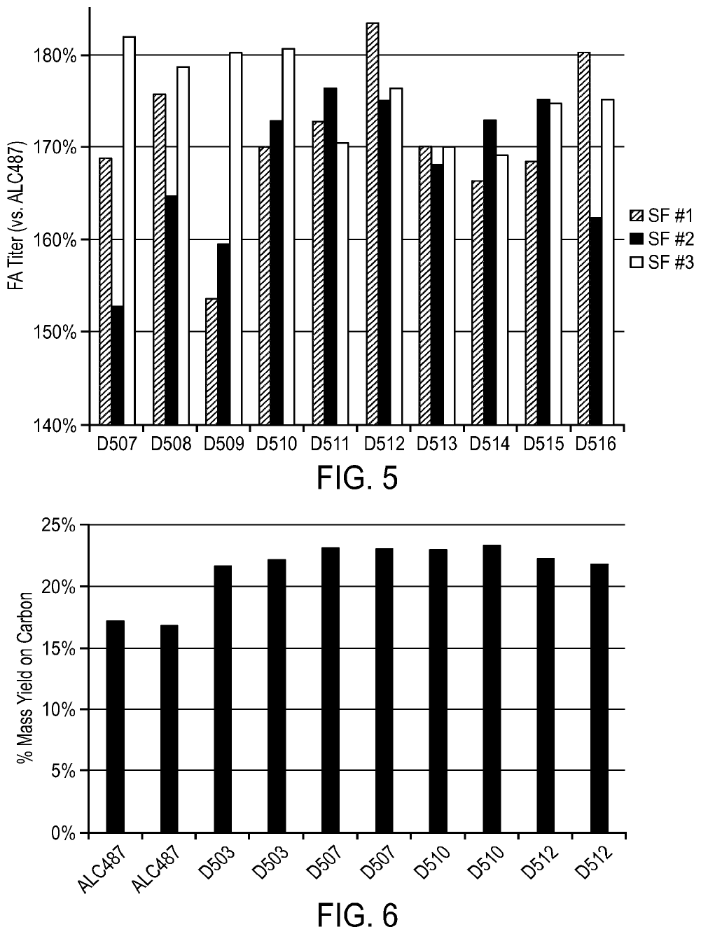

Several of the top producing strains expressing wild-type FadR identified in the initial screen were assigned strain IDs and validated in a shake flask fermentation. Briefly, each strain was streaked for single colonies, and three separate colonies from each strain were grown in three separate flasks according to the shake flask fermentation protocol described in Example 1. Total fatty species were measured using GC-FID as described in Example 1. All of the strains expressing wild-type FadR displayed higher total fatty species titers as compared to the control ALC487 strain (FIG. 5).

Several of the top producing strains expressing wild-type FadR were then further characterized in order to determine the yield of fatty species. To do so, a shake flask fermentation was performed as described above, except that (i) the temperature was held at 32° C., (ii) additional glucose was added after 18 hours and 43 hours, and (iii) extraction was performed at 68.5 hours. The total fatty species produced was divided by the total glucose consumed to calculate the fatty species yield. All of the strains expressing wild-type FadR displayed a higher yield of total fatty species as compared to the control ALC487 strain (FIG. 6).

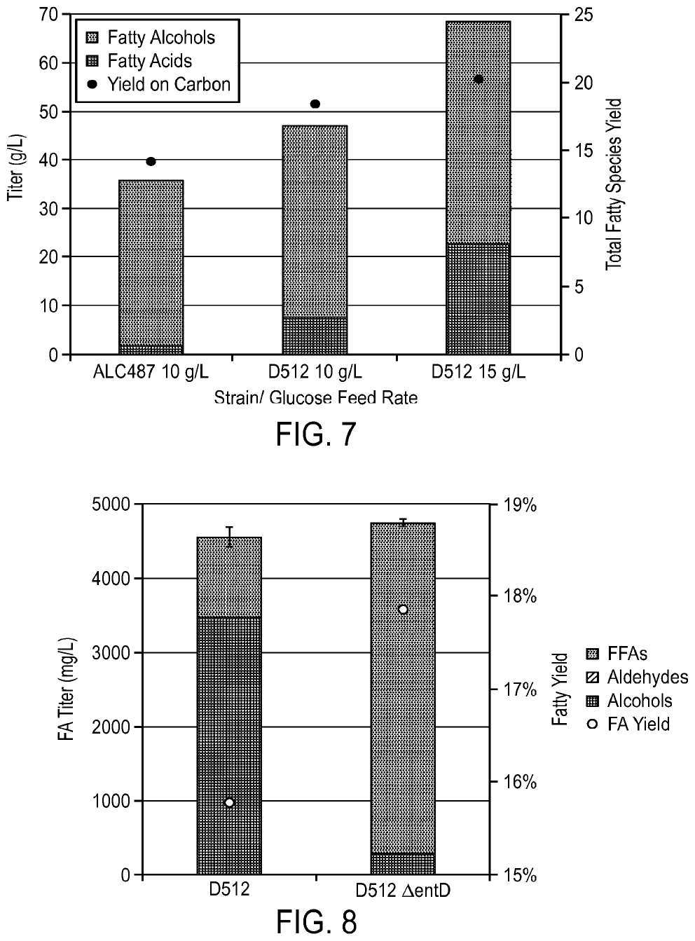

The D512 strain was then further characterized by evaluating total fatty species titer and yield following fermentation in a 5 L bioreactor. At a glucose feed rate of 10 g/L/hr glucose, the D512 strain produced higher titers of fatty acids and fatty alcohols as compared to the control ALC487 strain (FIG. 7). In addition, the total yield on all fatty species increased in the D512 strain as compared to the ALC487 strain (FIG. 7). At a higher glucose feed rate of 15 g/L/hr, the D512 strain produced approximately 68.5 g/L total fatty species at a yield of approximately 20% (FIG. 7). The D512 strain produced a higher total fatty species titer and yield at 15 g/L/hr as compared to 10 g/L/hr (FIG. 7).

Plasmid DNA was isolated from the D512 strain and sequenced according to standard protocols. The plasmid obtained from the D512 strain, termed pDG109, was determined to have the sequence corresponding to SEQ ID NO: 23.

The results of this example demonstrate that engineered host cells having an altered level of expression of FadR produce higher titers and yields of fatty acids and derivatives thereof as compared to corresponding wild-type host cells.

### EXAMPLE 4

This example demonstrates a method to produce high titers of fatty acids in engineered host cells having an altered level of expression of FadR.

The E. coli EG149 strain utilized in Example 3 overexpresses the entD gene, which encodes a phosphopantetheine transferase (PPTase) involved in the activation of the CarB enzyme that catalyzes the reduction of fatty acids to fatty aldehydes and fatty alcohols.

To assess the effect of entD expression on fatty acid and fatty alcohol production in the D512 strain, a D512 variant was generated which contained a deletion of the entD gene (D512 ΔentD). Shake flask fermentations were performed with the D512 strain and the D512 AentD strain as described in Example 1. The D512 strain produced high titers of fatty alcohols and comparatively lower titers and yields of fatty acids (FIG. 7). In contrast, the D512 ΔentD strain produced high titers and yields of fatty acids, and relatively low titers of fatty alcohols (FIG. 8). The titers of total fatty species were similar between the D512 strain and the D512 ΔentD strain (FIG. 8).

The results of this example demonstrate that engineered host cells having an altered level of FadR expression produce high titers of fatty acids when the entD gene is deleted.

### EXAMPLE 5

This example demonstrates a method to identify engineered host cells which display enhanced production of fatty acids and derivatives thereof.

To further assess the effect of altered FadR expression on the production of fatty acids and derivatives thereof, ribosome binding site (RBS) libraries of FadR (S219N) and wild-type FadR were prepared and screened in E. coli host cells.

An RBS library was inserted upstream of the fadR (S219N) gene in pDS57 as follows. The genomic DNA of a strain containing the fadR (S219N) allele Moniker stEP005; id: s26z7 was amplified by PCR using the DG191 (SEQ ID NO: 17) and fadR (S219N)_pme319rc (SEQ ID NO: 24) primer set. The primers used in this example are listed in Table 3.

TABLE 3

| | | Sequence |
|---|---|---|
| Primer | Sequence | Identifier |
| DG191 | ATGGTCATTAAGGCGCAAAGCCCGG | SEQ ID NO: 17 |
| fadR | CAAAACAGCCAAGCTGGAGACCGTTTTTATCGCC | SEQ ID NO: 24 |
| (S219N)_ | CCTGAATGGCTAAATCACC | |
| pme319rc | | |
| 377-rbs-fadR | GCCCGAACCCGCAAGTAANHHARNDDHDDNWAG | SEQ ID NO: 25 |
| (S219N)f | GARNNNNNNNATGGTCATTAAGGCGCAAAGCCC | |
| | GG | |
| NH246 | AAAAACGGTCTCCAGCTTGGCTGTTTTGGCGGAT | SEQ ID NO: 26 |
| | GAGAGAAGATTTTC | |
| 377-3r | TTACTTGCGGGTTCGGGCGC | SEQ ID NO: 27 |

After the fadR (S219N) template was made, the RBS was added by PCR using the 377-rbs-fadR (S219N)f (SEQ ID NO: 25) and fadR (S219N)-pme319rc (SEQ ID NO: 24) primer set. The 377-rbs-fadR (S219N) f primer contained degenerate nucleotides to introduce variability into the RBS library. The RBS-fadR (S219N) was ligated with a pDS57 vector backbone (described in Example 5), using the commercial available CLONEZ™ kit from Genscript (Piscataway, NJ) with the NH246 (SEQ ID NO: 26) and 377-3r (SEQ ID NO: 27) primer set.

An RBS library also was inserted upstream of the wild-type fadR gene in pDS57 using s similar protocol, except that the wild-type fadR gene was amplified by PCR using E. coli DV2 genomic DNA.

The ligated pDS57-rbs-fadR (S219N) and pDS57-rbs-fadR constructs were transformed separately into an E. coli DAM1 strain by electroporation. Strain DAM1 was produced as a derivative of strain DV2 (MG1655 ΔfadE, ΔfhuA), where the lacI^{q}-P_{Trc}-tesA-fadD genes were integrated into the chromosome using the Tn7-based delivery system present in plasmid pGRG25 (described in Mckenzie et al., BMC Microbiology 6:39 (2006)). After transformation, the cells were recovered for 1 hour at 37°° C. followed by plating on LB agar containing spectinomycin. After overnight incubation at 37° C., single colonies were picked to screen in 96 deep well-plates containing 300 μL/well LB with spectinomycin. The plates were incubated in a 32° C. shaker with 80% humidity and shaking at 250 RPM for approximately 5 hours. After 5 hours of growth, 30 μL/well of LB culture was transferred to 300 μL/well FA2 (2 g/L nitrogen) medium containing spectinomycin. Plates were incubated again in a 32° C. shaker with 80% humidity and shaking at 250 RPM overnight. 30 μL/well of the overnight culture was inoculated into 300 μL/well FA2 (1 g/L nitrogen) medium containing spectinomycin, 1 mM IPTG, and 2% methanol. One replicate plate was incubated in 32° C. shaker and another was incubated in a 37° C. shaker with 80% humidity and shaking at 250 RPM overnight. The recipe for FA2 medium is listed in Table 4.

TABLE 4

| Reagent | mL Reagent per 1000 mL FA2 |
|---|---|
| 5× Salt Solution | 200 |
| Thiamine (10 mg/mL) | 0.1 |
| 1M MgSO₄ | 1 |
| 1M CaCl₂ | 0.1 |
| 50% glucose | 60 |
| TM2 (trace minerals no iron) | 1 |
| 10 g/L ferric citrate | 1 |
| 2M Bis-Tris buffer | 50 |
| NH₄Cl* | 10 |
| Water | q.s. to 1000 mL |

*eliminated for FA2 (1 g/L nitrogen) medium

After approximately 24 hours of incubation, the plates were extracted by adding 40 μL/well 1M HCl and 300 μL/well butyl acetate. The plates were shaken for 15 minutes at 2000 RPM, and then centrifuged for 10 minutes at 4500 RPM at room temperature. 50 μL of the organic layer per well was transferred to a shallow well 96-well plate containing 50 μL/well BSTFA (Sigma Aldrich, St. Louis, MO), and the extracts were analyzed by GC-FID.

Several clones of E. coli DAMI transformed with pDS57-rbs-fadR were identified as producing substantially higher titers of FAMEs and free fatty acids as compared to control E. coli DAM1 transformed with pDS57 alone. In general, the FadR variants produced low C14 to C16 ratios and displayed overall higher titers at 32° C. as compared to 37° C.

Numerous clones of E. coli DAM1 transformed with pDS57-rbs-FadR (S219N) also were identified as producing substantially higher titers of FAMEs and free fatty acids as compared to control E. coli DV2 or E. coli DAM1 transformed with pDS57 alone.

Two of the clones transformed with pDS57-rbs-FadR (S219N) identified in the initial screen (designated as P1A4 and P1G7) were further characterized in shake flask fermentations. Briefly, each colony was inoculated into 5 mL of LB containing spectinomycin and incubated at 37° C. with shaking at approximately 200 RPM for about 5 hours. 1.5 mL of the LB culture was transferred into 13.5 mL FA2 (2 g/L nitrogen) medium containing 0.05% Triton X-100 and spectinomycin in a 125 mL baffled flask. The flask cultures were incubated overnight at 32° C., 80% humidity and 250 RPM. 1.5 mL of the overnight culture was transferred into a new 125 mL baffled flask that contained 13.5 mL FA2 (1 g/L nitrogen) medium containing 0.05% Triton X-100, 1 mM IPTG, 2% methanol, and spectinomycin. The flask cultures were then incubated at 32° C., 80% humidity and 250 RPM. After 56 hours of incubation, 500 μL samples were taken out from each flask. 100 μL of each sample was diluted with 900 μL water to measure the OD of the culture, 100 μL of each sample was diluted with 900 μL water to measure remaining glucose, and 300 μL of each sample was extracted and analyzed using GC-FID as described above.

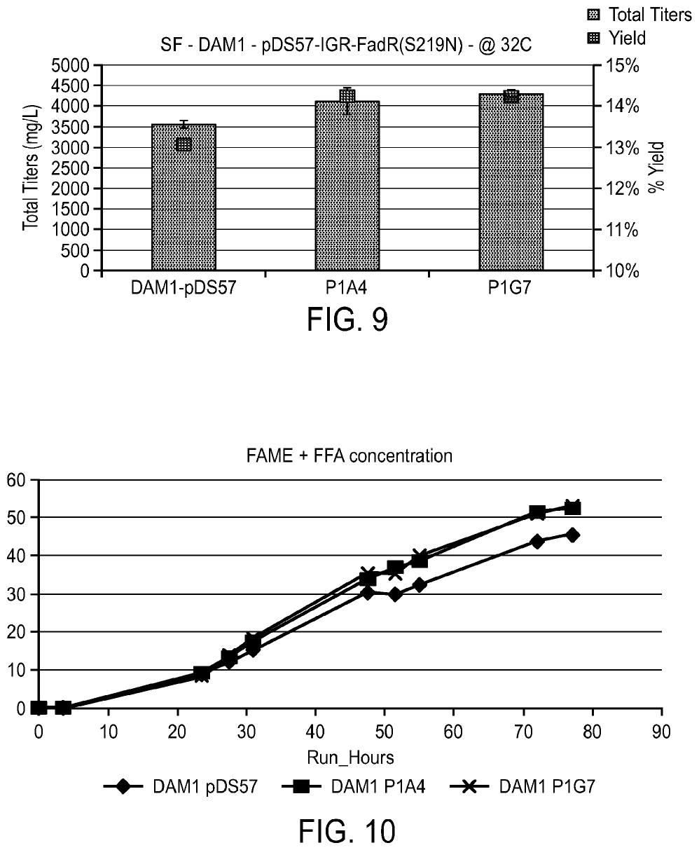

Both of the two FadR variants (i.e., P1A4 and P1G7) produced higher titers and yields of total fatty species as compared to the control strain transformed with pDS57 alone in the shake flask fermentation (FIG. 9).

Production of fatty species by P1A4 and P1G7 was also measured in large scale fermentations. To do so, cells from a frozen stock were grown in LB media for a few hours and then transferred to a defined media consisting of 3 g/L KH₂PO₄, 6 g/L Na₂HPO₄ dihydrate, 2 g/L NH₄Cl, 0.24 g/L MgSO₄×7 H₂O, 20 g/L glucose, 200 mM Bis-Tris buffer (pH 7.2), 1.0 ml/L trace metals solution, and 1.0 mg/L thiamine, and cultured overnight. The trace metals solution was composed of 27 g/L FeCl₃×6 H₂O, 2 g/L ZnCl₂×4H₂O, 2 g/L CaCl₂×6 H₂O, 2 g/L Na₂MoO₄×2H₂O, 1.9 g/L CuSO₄×5 H₂O, 0.5 g/L H₃BO₃, and 40 mL/L of concentrated HCl.

50 mL of each overnight culture was inoculated into 1 liter of production medium in a fermentor with temperature, pH, agitation, aeration and dissolved oxygen control. The medium composition was as follows: 1 g/L KH₂PO₄, 0.5 g/L (NH₄) 2SO₄, 0.5 g/L MgSO₄×7 H₂O, 5 g/L Bacto casaminoacids, 0.034 g/L ferric citrate, 0.12 ml/L 1M HCl, 0.02 g/L ZnCl₂×4 H₂O, 0.02 g/L CaCl₂×2H₂O, 0.02 g/L Na₂MoO₄×2H₂O, 0.019 g/L CuSO₄×5 H₂O, 0.005 g/L H₃BO₃ and 1.25 mL/L of a vitamin solution. The vitamin solution contained 0.06 g/L riboflavina, 5.40 g/L pantothenic acid, 6.0 g/L niacine, 1.4 g/L piridoxine and 0.01 g/L folic acid.

The fermentations were performed at 32° C., pH 6.8, and dissolved oxygen (DO) equal to 25% of saturation. pH was maintained by addition of NH₄OH, which also served as nitrogen source for cell growth. When the initial 5 g/L of glucose had been almost consumed, a feed consisting of 500g/L glucose, 1.6 g/L of KH₂PO₄, 3.9 g/L MgSO₄·7H₂O, 0.13 g/L ferric citrate, and 30 ml/L of methanol was supplied to the fermentor.

In the early phase of growth, the production of FAME was induced by the addition of 1 mM IPTG and 20 ml/L of pure methanol. After most of the cell growth was completed, the feed rate was maintained at a rate of 7.5 g glucose/L/hour. After induction, methanol was continuously supplied with the glucose feed. The fermentation was continued for a period of 3 days, and samples were taken at several timepoints for analysis of fatty species as described above.

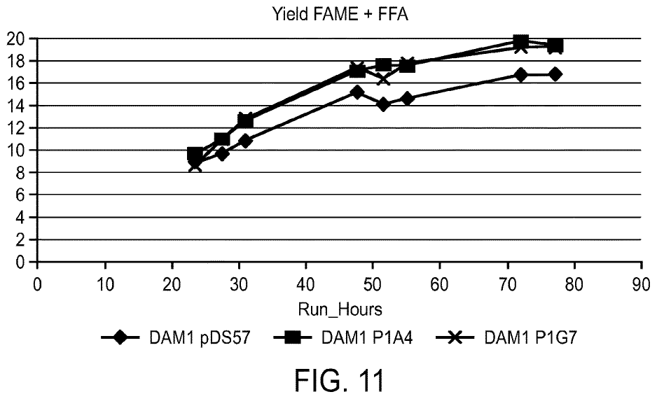

P1A4 and P1G7 produced higher titers of total fatty species (FAMEs and free fatty acids) as compared to the control strain transformed with pDS57 alone in the large scale fermentations (FIG. 10). In addition, P1A4 and P1G7 produced higher yields of total fatty species as compared to the control strain in the large scale fermentations (FIG. 11).

The results of this example demonstrate methods to produce engineered host cells having altered FadR expression which display enhanced titers and yields of FAMEs and fatty acids as compared to corresponding wild-type cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-46. (canceled)

47. A recombinant host cell which is genetically engineered to have an increased level of expression of a FadR polypeptide as compared to the level of expression of the FadR polypeptide in a corresponding wild-type host cell,
wherein one or more of the titer, yield, or productivity of a fatty acid or a derivative thereof produced by the recombinant host cell is increased relative to that of a corresponding wild-type host cell when the recombinant host cell is cultured in a culture medium under conditions permissive for the production of the fatty acid or derivative thereof.

48. The recombinant host cell of claim 47, wherein:
the fatty acid or derivative thereof is an acyl-ACP, an acyl-CoA, a fatty acid, a fatty aldehyde, a short chain alcohol, a long chain alcohol, a fatty alcohol, a wax ester, a fatty ester, a fatty acid methyl ester (FAME), a fatty acid ethyl ester (FAEE), a hydrocarbon, a ketone, a terminal olefin, or an internal olefin;
the fatty acid or derivative thereof is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty acid or derivative thereof;
the fatty acid or derivative thereof is saturated or unsaturated; and
the fatty acid or derivative thereof is branched or unbranched.

49. The recombinant host cell of claim 47, wherein the FadR polypeptide is encoded by a fadR gene obtained from Escherichia, Salmonella, Citrobacter, Enterobacter, Klebsiella, Cronobacter, Yersinia, Serratia, Erwinia, Pectobacterium, Photorhabdus, Edwardsiella, Shewanella, or Vibrio.

50. The recombinant host cell of claim 47, wherein the FadR polypeptide is a wild-type FadR polypeptide or a mutant FadR polypeptide.

51. The recombinant host cell of claim 47, wherein the FadR polypeptide comprises an amino acid sequence having at least 80% sequence identity to the amino acid sequence of SEQ ID NO: 1.

52. The recombinant host cell of claim 50, wherein the mutant FadR polypeptide comprises an amino acid sequence having at least 80% sequence identity to SEQ ID NO: 1 and comprises a substitution mutation at position 219 of SEQ ID NO: 1, wherein the amino acid residue corresponding to amino acid 219 of SEQ ID NO: 1 is substituted with an asparagine residue.

53. The recombinant host cell of claim 47, wherein the level of expression of the FadR polypeptide in the recombinant host cell is regulated by an expression control sequence operably linked to a polynucleotide sequence which encodes the FadR polypeptide.

54. The recombinant host cell of claim 53, wherein the expression control sequence is a promoter or a ribosome binding site.

55. The recombinant host cell of claim 47, wherein the recombinant host cell further comprises an altered level of expression of one or more genes corresponding to fabA, fabB, iclR, fadA, fadB, fadD, fadE, fadl, fadJ, fadL, fadM, uspA, aceA, aceB, and aceK, as compared to the level of expression of the gene(s) in a corresponding wild-type host cell.

56. The recombinant host cell of claim 47, wherein the recombinant host cell further expresses or overexpresses one or more of a thioesterase, a carboxylic acid reductase (CAR), an alcohol dehydrogenase, an ester synthase, a decarboxylase, an aldehyde decarbonylase, a fatty alcohol forming acyl-CoA reductase (FAR), an acyl-ACP reductase, an acyl-CoA reductase, OleA, OleCD, OleBCD, an acetyl-CoA carboxylase complex, a biotin protein ligase, or a phosphopantetheinyl transferase (PPTase).

57. The recombinant host cell of claim 56, wherein the acetyl-CoA carboxylase complex is encoded by one or more acetyl-CoA carboxylase subunit genes obtained from one or more of Corynebacterium glutamicum, Escherichia coli, Lactococcus lactis, Kineococcus radiotolerans, Desulfovibrio desulfuricans, Erwinia amylovora, Rhodospirillum rubrum, Vibrio furnissii, Stenotrophomonas maltophilia, Synechocystis sp. PCC6803, or Synechococcus elongatus.

58. The recombinant host cell of claim 56, wherein:
(a) a thioesterase is expressed or overexpressed, and the recombinant host cell produces a fatty acid;
(b) a thioesterase and a carboxylic acid reductase are expressed or overexpressed, and the recombinant host cell produces a fatty aldehyde or a fatty alcohol or a combination thereof;
(c) a thioesterase, a carboxylic acid reductase, and an alcohol dehydrogenase are expressed or overexpressed, and the recombinant host cell produces a fatty alcohol;
(d) a thioesterase, a carboxylic acid reductase, and a phosphopantetheinyl transferase (PPTase) are expressed or overexpressed, and the recombinant host cell produces a fatty alcohol;
(e) a thioesterase, a carboxylic acid reductase, an alcohol dehydrogenase, and a phosphopantetheinyl transferase (PPTase) are expressed or overexpressed, and the recombinant host cell produces a fatty alcohol; or
(f) an ester synthase is expressed or overexpressed, and the recombinant host cell produces a fatty ester or a wax ester.

59. A method of producing a fatty acid or a derivative thereof, the method comprising culturing the recombinant host cell of claim 47 in a culture medium under conditions permissive for the production of the fatty acid or derivative thereof.

60. The method of claim 59, further comprising isolating the fatty acid or derivative thereof.

61. The method of claim 59, wherein the FadR polypeptide comprises an amino acid sequence having at least 80% sequence identity to the amino acid sequence of SEQ ID NO: 1.

62. The method of claim 59, wherein the FadR polypeptide comprises an amino acid sequence having at least 80% sequence identity to SEQ ID NO: 1 and comprises a substitution mutation at position 219 of SEQ ID NO: 1, wherein the amino acid residue corresponding to amino acid 219 of SEQ ID NO: 1 is substituted with an asparagine residue.

63. The method of claim 59, wherein:
(a) the FadR polypeptide is a wild-type FadR polypeptide;
(b) the FadR polypeptide is a mutant FadR polypeptide; or
(c) the FadR polypeptide is encoded by a fadR gene obtained from Escherichia, Salmonella, Citrobacter, Enterobacter, Klebsiella, Cronobacter, Yersinia, Serratia, Erwinia, Pectobacterium, Photorhabdus, Edwardsiella, Shewanella, or Vibrio.

64. The method of claim 59, wherein the level of expression of the FadR polypeptide in the recombinant host cell is regulated by an expression control sequence operably linked to a polynucleotide sequence which encodes the FadR polypeptide.

65. The method of claim 64, wherein the expression control sequence is a promoter or a ribosome binding site.

66. The method of claim 59, wherein the recombinant host cell further expresses or overexpresses one or more of a thioesterase, a carboxylic acid reductase (CAR), an alcohol dehydrogenase, an ester synthase, a decarboxylase, an aldehyde decarbonylase, a fatty alcohol forming acyl-CoA reductase (FAR), an acyl-ACP reductase, an acyl-CoA reductase, OleA, OleCD, OleBCD, one or more acetyl-CoA carboxylase complex subunit genes, a biotin protein ligase, or a phosphopantetheinyl transferase (PPTase).

67. The method of claim 59, wherein:
the fatty acid or derivative thereof is an acyl-ACP, an acyl-CoA, a fatty acid, a fatty aldehyde, a short chain alcohol, a long chain alcohol, a fatty alcohol, a wax ester, a fatty ester, a fatty acid methyl ester (FAME), a fatty acid ethyl ester (FAEE), a hydrocarbon, a ketone, a terminal olefin, or an internal olefin;
the fatty acid or derivative thereof is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty acid or derivative thereof;
the fatty acid or derivative thereof is saturated or unsaturated; and
the fatty acid or derivative thereof is branched or unbranched.

68. The method of claim 59, wherein the recombinant host cell further comprises an altered level of expression of one or more genes corresponding to fabA, fabB, icIR, fadA, fadB, fadD, fadE, fadI, fadJ, fadL, fadM, uspA, aceA, aceB, and aceK, as compared to the level of expression of the gene(s) in a corresponding wild-type host cell.
